SEQUENCE LISTING (21 sequences)

SEQ ID NO: 1 - DNA, 660 bp, synthetic construct
  source 1..660: mol_type = other DNA
atggattaccccgaggcagtggattggcgcactaagggggcagttactgcggtgaaggatcagggagactgtggtgcatcatacgcatttagcgccatgggtgccttggaaggcgcgaacgccttagcgaaaggcaacgccgtatccctgtcggagcaaaacatcattgattgttcaattccttatggtaaccatggttgtcatggtggaaacatgtatgatgctttcttatacgtcatcgcgaatgagggagtggaccaagattcagcgtatcctttcgtcgggaaacagtcctcttgtaactacaactcgaagtataagggcacgtcgatgagtggtatggtgtctattaagtcgggatcagagtccgatttgcaagcagccgtctctaatgtcggcccagtttcggtggcaattgacggcgcaaactcggccttccgcttttattattctggcgtctatgattccagccgttgttctagttcgtctttgaatcatgcaatggtggtgacggggtacggcagttacaatggaaaaaaatactggcttgccaagaactcctggggcactaactgggggaactcagggtacgttatgatggctcgtaataaatacaatcagtgcggtattgcgacggacgcatcctatcctacgttgtaa

SEQ ID NO: 2 - protein, 219 aa, synthetic construct
MDYPEAVDWRTKGAVTAVKDQGDCGASYAFSAMGALEGANALAKGNAVSLSEQNIIDCSIPYGNHGCHGGNMYDAFLYVIANEGVDQDSAYPFVGKQSSCNYNSKYKGTSMSGMVSIKSGSESDLQAAVSNVGPVSVAIDGANSAFRFYYSGVYDSSRCSSSSLNHAMVVTGYGSYNGKKYWLAKNSWGTNWGNSGYVMMARNKYNQCGIATDASYPTL

SEQ ID NO: 3 - DNA, 993 bp, synthetic construct
  source 1..993: mol_type = other DNA
atgtatctcggcacgttggttgttttgtgtgttttgggggctgctattggagagccaatgcctcagtatgagttcaaggaggaatggcagctgtggaagaaacaacatgacaagtcttacagcaccaacttggaggaactggagaaacatcttgtctggctctccaacaagaagtacattgaactgcacaatgccaatgcagacacctttggattcactctagctatgaaccatctaggagatatgactgaccatgaatacaaggagagatacctcacatacactaacagcaaatctggtaactacaccaaggtgttcaaacgtgagccatggatggcctacccggagactgtagattggagaacaaagggcgctgtgactggtatcaagagccagggagattgtggtgccagctatgcattcagtgccatgggtgcacttgaaggaatcaatgcacttgctactggaaagctgacctatctcagtgaacagaacatcattgattgctctgtaccttatggtaaccatggttgcaagggtggaaacatgtatgtggctttcctctatgttgttgctaacgaaggagttgatgatgggggttcctatccatttagaggaaagcaatccagttgtacgtatcaagagcagtaccgtggtgcaagtatgtctggctcagttcaaatcaacagtggtagtgaatctgatctggaagcagctgtagccaatgttggtccagttgcagtagctattgatggagagtcaaatgctttcagattctattacagtggagtgtacgactcctccagatgttctagtagcagtctcaaccacgccatggtgatcactggctatggaatttcaaataaccaggaatactggcttgcaaagaacagctggggtgagaactggggagaactgggctatgtgaagatggccaggaacaagtacaatcaatgtgggattgctagtgatgcctcctaccccactctctag

SEQ ID NO: 4 - protein, 330 aa, synthetic construct
MYLGTLVVLCVLGAAIGEPMPQYEFKEEWQLWKKQHDKSYSTNLEELEKHLVWLSNKKYIELHNANADTFGFTLAMNHLGDMTDHEYKERYLTYTNSKSGNYTKVFKREPWMAYPETVDWRTKGAVTGIKSQGDCGASYAFSAMGALEGINALATGKLTYLSEQNIIDCSVPYGNHGCKGGNMYVAFLYVVANEGVDDGGSYPFRGKQSSCTYQEQYRGASMSGSVQINSGSESDLEAAVANVGPVAVAIDGESNAFRFYYSGVYDSSRCSSSSLNHAMVITGYGISNNQEYWLAKNSWGENWGELGYVKMARNKYNQCGIASDASYPTL

SEQ ID NO: 5 - DNA, 462 bp, synthetic construct
  source 1..462: mol_type = other DNA
atgaaggctacaaaattggtgttgggagcggtaatcctgggttcgactctgttggcaggctgttcttccaatgcgaagatcgaccagttatccagtgatgtggggatcaacccatacgtcggtttcgagatgggttatgactggctgggccgcatgccatataagggatccgtggaaaacggcgcatataaggcacaaggagtccaattgaccgcaaagttgggttaccccattactgatgatttggacatctacacgcgtctgggtgggatggtgtggcgcgcagacacaaaaagtaatgtttatggtaagaatcacgatacaggagtttctcccgtttttgctggaggagtggagtatgcgatcacgccggaaatcgcgacgcgcttggagtaccaatggactaacaacattggcgacgcgcacacgattggcactcgtcctgacaacggcatcccaggt

SEQ ID NO: 6 - protein, 154 aa, synthetic construct
MKATKLVLGAVILGSTLLAGCSSNAKIDQLSSDVGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGIPG

SEQ ID NO: 7 - DNA, 732 bp, synthetic construct
  source 1..732: mol_type = other DNA
atgaccttagataaagccttggtattacgcacgtgtgctaacaatatggcagatcactgcgggttaatttggccggccagcggtactgtggagagccgctattggcagagtacccgccgccacgaaaacgggcttgttggccttctgtggggagccggtacgtctgcctttctgtccgtccatgccgatgcgcgttggatcgtctgcgaagtcgccgtagcggacatcatttcgttggaggaaccgggcatggtcaaattcccccgcgctgaagttgttcacgtgggtgatcgcatttcagcttcacacttcatctcggcacgccaagccgatcccgcaagcacgagcaccagcacttcaacttccaccttaacgcctatgccgaccgcgattccgacaccgatgccggccgttgcatcggttactctgccggtcgccgagcaggcccgccatgaagttttcgacgtagcgagtgtttcggccgccgctgcccccgtgaatactctgcctgtcactacgcctcagaaccttcagactgcgacttacgggagcacattatctggggataaccactctcgcttaatcttccgtttgtgggacggtaaacgttatcgccaacttgtggctcgtaccggcgagaacggggttgaggctgacatcccgtactatgtgaacgaggacgacgatattgttgataagccggacgaggacgacgactggatcgaggtgaag

SEQ ID NO: 8 - protein, 244 aa, synthetic construct
MTLDKALVLRTCANNMADHCGLIWPASGTVESRYWQSTRRHENGLVGLLWGAGTSAFLSVHADARWIVCEVAVADIISLEEPGMVKFPRAEVVHVGDRISASHFISARQADPASTSTSTSTSTLTPMPTAIPTPMPAVASVTLPVAEQARHEVFDVASVSAAAAPVNTLPVTTPQNLQTATYGSTLSGDNHSRLIFRLWDGKRYRQLVARTGENGVEADIPYYVNEDDDIVDKPDEDDDWIEVK

SEQ ID NO: 9 - DNA, 31 bp, synthetic construct
  source 1..31: mol_type = other DNA
tttacactttatgcttccggctcgtataatg

SEQ ID NO: 10 - DNA, 119 bp, synthetic construct
  source 1..119: mol_type = other DNA
caccacaattcagcaaattgtgaacatcatcacgttcatctttccctggttgccaatggcccattttcctgtcagtaacgagaaggtcgcgaattcaggcgctttttagactggtcgta

SEQ ID NO: 11 - DNA, 12 bp, synthetic construct
  source 1..12: mol_type = other DNA
aaagaggagaaa

SEQ ID NO: 12 - DNA, 115 bp, synthetic construct
  source 1..115: mol_type = other DNA
caaataaaacgaaaggctcagtcgaaagactgggcctttcgttttatctgttgtttgtcggtgaacgctctctactagagtcacactggctcaccttcgggtgggcctttctgcg

SEQ ID NO: 13 - DNA, 1296 bp, synthetic construct
  source 1..1296: mol_type = other DNA
caccacaattcagcaaattgtgaacatcatcacgttcatctttccctggttgccaatggcccattttcctgtcagtaacgagaaggtcgcgaattcaggcgctttttagactggtcgtaatgaaattcaggaggttgtcgactctagatactagagaaagaggagaaatactagatgaaggctacaaaattggtgttgggagcggtaatcctgggttcgactctgttggcaggctgttcttccaatgcgaagatcgaccagttatccagtgatgtggggatcaacccatacgtcggtttcgagatgggttatgactggctgggccgcatgccatataagggatccgtggaaaacggcgcatataaggcacaaggagtccaattgaccgcaaagttgggttaccccattactgatgatttggacatctacacgcgtctgggtgggatggtgtggcgcgcagacacaaaaagtaatgtttatggtaagaatcacgatacaggagtttctcccgtttttgctggaggagtggagtatgcgatcacgccggaaatcgcgacgcgcttggagtaccaatggactaacaacattggcgacgcgcacacgattggcactcgtcctgacaacggcatcccaggtatggattaccccgaggcagtggattggcgcactaagggggcagttactgcggtgaaggatcagggagactgtggtgcatcatacgcatttagcgccatgggtgccttggaaggcgcgaacgccttagcgaaaggcaacgccgtatccctgtcggagcaaaacatcattgattgttcaattccttatggtaaccatggttgtcatggtggaaacatgtatgatgctttcttatacgtcatcgcgaatgagggagtggaccaagattcagcgtatcctttcgtcgggaaacagtcctcttgtaactacaactcgaagtataagggcacgtcgatgagtggtatggtgtctattaagtcgggatcagagtccgatttgcaagcagccgtctctaatgtcggcccagtttcggtggcaattgacggcgcaaactcggccttccgcttttattattctggcgtctatgattccagccgttgttctagttcgtctttgaatcatgcaatggtggtgacggggtacggcagttacaatggaaaaaaatactggcttgccaagaactcctggggcactaactgggggaactcagggtacgttatgatggctcgtaataaatacaatcagtgcggtattgcgacggacgcatcctatcctacgttgtaa

SEQ ID NO: 14 - protein, 373 aa, synthetic construct
MKATKLVLGAVILGSTLLAGCSSNAKIDQLSSDVGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGIPGMDYPEAVDWRTKGAVTAVKDQGDCGASYAFSAMGALEGANALAKGNAVSLSEQNIIDCSIPYGNHGCHGGNMYDAFLYVIANEGVDQDSAYPFVGKQSSCNYNSKYKGTSMSGMVSIKSGSESDLQAAVSNVGPVSVAIDGANSAFRFYYSGVYDSSRCSSSSLNHAMVVTGYGSYNGKKYWLAKNSWGTNWGNSGYVMMARNKYNQCGIATDASYPTL

SEQ ID NO: 15 - DNA, 6952 bp, synthetic construct
  source 1..6952: mol_type = other DNA
ttctcatgtttgacagcttatcatcgataagcttaattaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaaattcaggaggttgtcgactctagatactagagaaagaggagaaatactagatgaaggctacaaaattggtgttgggagcggtaatcctgggttcgactctgttggcaggctgttcttccaatgcgaagatcgaccagttatccagtgatgtggggatcaacccatacgtcggtttcgagatgggttatgactggctgggccgcatgccatataagggatccgtggaaaacggcgcatataaggcacaaggagtccaattgaccgcaaagttgggttaccccattactgatgatttggacatctacacgcgtctgggtgggatggtgtggcgcgcagacacaaaaagtaatgtttatggtaagaatcacgatacaggagtttctcccgtttttgctggaggagtggagtatgcgatcacgccggaaatcgcgacgcgcttggagtaccaatggactaacaacattggcgacgcgcacacgattggcactcgtcctgacaacggcatcccaggtatggattaccccgaggcagtggattggcgcactaagggggcagttactgcggtgaaggatcagggagactgtggtgcatcatacgcatttagcgccatgggtgccttggaaggcgcgaacgccttagcgaaaggcaacgccgtatccctgtcggagcaaaacatcattgattgttcaattccttatggtaaccatggttgtcatggtggaaacatgtatgatgctttcttatacgtcatcgcgaatgagggagtggaccaagattcagcgtatcctttcgtcgggaaacagtcctcttgtaactacaactcgaagtataagggcacgtcgatgagtggtatggtgtctattaagtcgggatcagagtccgatttgcaagcagccgtctctaatgtcggcccagtttcggtggcaattgacggcgcaaactcggccttccgcttttattattctggcgtctatgattccagccgttgttctagttcgtctttgaatcatgcaatggtggtgacggggtacggcagttacaatggaaaaaaatactggcttgccaagaactcctggggcactaactgggggaactcagggtacgttatgatggctcgtaataaatacaatcagtgcggtattgcgacggacgcatcctatcctacgttgtaaggtacctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 16 - DNA, 1685 bp, synthetic construct
  source 1..1685: mol_type = other DNA
tttacactttatgcttccggctcgtataatgtgtggaattgtgagcggataacaatttcagaattctactagagaaagaggagaaatactagatgaaggctacaaaattggtgttgggagcggtaatcctgggttcgactctgttggcaggctgttcttccaatgcgaagatcgaccagttatccagtgatgtggggatcaacccatacgtcggtttcgagatgggttatgactggctgggccgcatgccatataagggatccgtggaaaacggcgcatataaggcacaaggagtccaattgaccgcaaagttgggttaccccattactgatgatttggacatctacacgcgtctgggtgggatggtgtggcgcgcagacacaaaaagtaatgtttatggtaagaatcacgatacaggagtttctcccgtttttgctggaggagtggagtatgcgatcacgccggaaatcgcgacgcgcttggagtaccaatggactaacaacattggcgacgcgcacacgattggcactcgtcctgacaacggcatcccaggtatgtatctcggcacgttggttgttttgtgtgttttgggggctgctattggagagccaatgcctcagtatgagttcaaggaggaatggcagctgtggaagaaacaacatgacaagtcttacagcaccaacttggaggaactggagaaacatcttgtctggctctccaacaagaagtacattgaactgcacaatgccaatgcagacacctttggattcactctagctatgaaccatctaggagatatgactgaccatgaatacaaggagagatacctcacatacactaacagcaaatctggtaactacaccaaggtgttcaaacgtgagccatggatggcctacccggagactgtagattggagaacaaagggcgctgtgactggtatcaagagccagggagattgtggtgccagctatgcattcagtgccatgggtgcacttgaaggaatcaatgcacttgctactggaaagctgacctatctcagtgaacagaacatcattgattgctctgtaccttatggtaaccatggttgcaagggtggaaacatgtatgtggctttcctctatgttgttgctaacgaaggagttgatgatgggggttcctatccatttagaggaaagcaatccagttgtacgtatcaagagcagtaccgtggtgcaagtatgtctggctcagttcaaatcaacagtggtagtgaatctgatctggaagcagctgtagccaatgttggtccagttgcagtagctattgatggagagtcaaatgctttcagattctattacagtggagtgtacgactcctccagatgttctagtagcagtctcaaccacgccatggtgatcactggctatggaatttcaaataaccaggaatactggcttgcaaagaacagctggggtgagaactggggagaactgggctatgtgaagatggccaggaacaagtacaatcaatgtgggattgctagtgatgcctcctaccccactctctagctcgagtaaggatctccaggcatcaaataaaacgaaaggctcagtcgaaagactgggcctttcgttttatctgttgtttgtcggtgaacgctctctactagagtcacactggctcaccttcgggtgggcctttctgcg

SEQ ID NO: 17 - protein, 484 aa, synthetic construct
MKATKLVLGAVILGSTLLAGCSSNAKIDQLSSDVGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGIPGMYLGTLVVLCVLGAAIGEPMPQYEFKEEWQLWKKQHDKSYSTNLEELEKHLVWLSNKKYIELHNANADTFGFTLAMNHLGDMTDHEYKERYLTYTNSKSGNYTKVFKREPWMAYPETVDWRTKGAVTGIKSQGDCGASYAFSAMGALEGINALATGKLTYLSEQNIIDCSVPYGNHGCKGGNMYVAFLYVVANEGVDDGGSYPFRGKQSSCTYQEQYRGASMSGSVQINSGSESDLEAAVANVGPVAVAIDGESNAFRFYYSGVYDSSRCSSSSLNHAMVITGYGISNNQEYWLAKNSWGENWGELGYVKMARNKYNQCGIASDASYPTL

SEQ ID NO: 18 - DNA, 6561 bp, synthetic construct
  source 1..6561: mol_type = other DNA
gacgtcggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgtttaggcaccccaggctttacactttatgcttccggctcgtataatgtgtggaattgtgagcggataacaatttcagaattctactagagaaagaggagaaatactagatgaaggctacaaaattggtgttgggagcggtaatcctgggttcgactctgttggcaggctgttcttccaatgcgaagatcgaccagttatccagtgatgtggggatcaacccatacgtcggtttcgagatgggttatgactggctgggccgcatgccatataagggatccgtggaaaacggcgcatataaggcacaaggagtccaattgaccgcaaagttgggttaccccattactgatgatttggacatctacacgcgtctgggtgggatggtgtggcgcgcagacacaaaaagtaatgtttatggtaagaatcacgatacaggagtttctcccgtttttgctggaggagtggagtatgcgatcacgccggaaatcgcgacgcgcttggagtaccaatggactaacaacattggcgacgcgcacacgattggcactcgtcctgacaacggcatcccaggtatgtatctcggcacgttggttgttttgtgtgttttgggggctgctattggagagccaatgcctcagtatgagttcaaggaggaatggcagctgtggaagaaacaacatgacaagtcttacagcaccaacttggaggaactggagaaacatcttgtctggctctccaacaagaagtacattgaactgcacaatgccaatgcagacacctttggattcactctagctatgaaccatctaggagatatgactgaccatgaatacaaggagagatacctcacatacactaacagcaaatctggtaactacaccaaggtgttcaaacgtgagccatggatggcctacccggagactgtagattggagaacaaagggcgctgtgactggtatcaagagccagggagattgtggtgccagctatgcattcagtgccatgggtgcacttgaaggaatcaatgcacttgctactggaaagctgacctatctcagtgaacagaacatcattgattgctctgtaccttatggtaaccatggttgcaagggtggaaacatgtatgtggctttcctctatgttgttgctaacgaaggagttgatgatgggggttcctatccatttagaggaaagcaatccagttgtacgtatcaagagcagtaccgtggtgcaagtatgtctggctcagttcaaatcaacagtggtagtgaatctgatctggaagcagctgtagccaatgttggtccagttgcagtagctattgatggagagtcaaatgctttcagattctattacagtggagtgtacgactcctccagatgttctagtagcagtctcaaccacgccatggtgatcactggctatggaatttcaaataaccaggaatactggcttgcaaagaacagctggggtgagaactggggagaactgggctatgtgaagatggccaggaacaagtacaatcaatgtgggattgctagtgatgcctcctaccccactctctagctcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggtgatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 19 - DNA, 1646 bp, synthetic construct
  source 1..1646: mol_type = other DNA
tttacactttatgcttccggctcgtataatgtgtggaattgtgagcggataacaatttcagaattctactagagaaagaggagaaatactagatgaccttagataaagccttggtattacgcacgtgtgctaacaatatggcagatcactgcgggttaatttggccggccagcggtactgtggagagccgctattggcagagtacccgccgccacgaaaacgggcttgttggccttctgtggggagccggtacgtctgcctttctgtccgtccatgccgatgcgcgttggatcgtctgcgaagtcgccgtagcggacatcatttcgttggaggaaccgggcatggtcaaattcccccgcgctgaagttgttcacgtgggtgatcgcatttcagcttcacacttcatctcggcacgccaagccgatcccgcaagcacgagcaccagcacttcaacttccaccttaacgcctatgccgaccgcgattccgacaccgatgccggccgttgcatcggttactctgccggtcgccgagcaggcccgccatgaagttttcgacgtagcgagtgtttcggccgccgctgcccccgtgaatactctgcctgtcactacgcctcagaaccttcagactgcgacttacgggagcacattatctggggataaccactctcgcttaatcttccgtttgtgggacggtaaacgttatcgccaacttgtggctcgtaccggcgagaacggggttgaggctgacatcccgtactatgtgaacgaggacgacgatattgttgataagccggacgaggacgacgactggatcgaggtgaagggaggaggcagtggcggtggaagtatggattaccccgaggcagtggattggcgcactaagggggcagttactgcggtgaaggatcagggagactgtggtgcatcatacgcatttagcgccatgggtgccttggaaggcgcgaacgccttagcgaaaggcaacgccgtatccctgtcggagcaaaacatcattgattgttcaattccttatggtaaccatggttgtcatggtggaaacatgtatgatgctttcttatacgtcatcgcgaatgagggagtggaccaagattcagcgtatcctttcgtcgggaaacagtcctcttgtaactacaactcgaagtataagggcacgtcgatgagtggtatggtgtctattaagtcgggatcagagtccgatttgcaagcagccgtctctaatgtcggcccagtttcggtggcaattgacggcgcaaactcggccttccgcttttattattctggcgtctatgattccagccgttgttctagttcgtctttgaatcatgcaatggtggtgacggggtacggcagttacaatggaaaaaaatactggcttgccaagaactcctggggcactaactgggggaactcagggtacgttatgatggctcgtaataaatacaatcagtgcggtattgcgacggacgcatcctatcctacgttgtaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcg

SEQ ID NO: 20 - protein, 471 aa, synthetic construct
MTLDKALVLRTCANNMADHCGLIWPASGTVESRYWQSTRRHENGLVGLLWGAGTSAFLSVHADARWIVCEVAVADIISLEEPGMVKFPRAEVVHVGDRISASHFISARQADPASTSTSTSTSTLTPMPTAIPTPMPAVASVTLPVAEQARHEVFDVASVSAAAAPVNTLPVTTPQNLQTATYGSTLSGDNHSRLIFRLWDGKRYRQLVARTGENGVEADIPYYVNEDDDIVDKPDEDDDWIEVKGGGSGGGSMDYPEAVDWRTKGAVTAVKDQGDCGASYAFSAMGALEGANALAKGNAVSLSEQNIIDCSIPYGNHGCHGGNMYDAFLYVIANEGVDQDSAYPFVGKQSSCNYNSKYKGTSMSGMVSIKSGSESDLQAAVSNVGPVSVAIDGANSAFRFYYSGVYDSSRCSSSSLNHAMVVTGYGSYNGKKYWLAKNSWGTNWGNSGYVMMARNKYNQCGIATDASYPTL

SEQ ID NO: 21 - DNA, 6522 bp, synthetic construct
  source 1..6522: mol_type = other DNA
gacgtcggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgtttaggcaccccaggctttacactttatgcttccggctcgtataatgtgtggaattgtgagcggataacaatttcagaattctactagagaaagaggagaaatactagatgaccttagataaagccttggtattacgcacgtgtgctaacaatatggcagatcactgcgggttaatttggccggccagcggtactgtggagagccgctattggcagagtacccgccgccacgaaaacgggcttgttggccttctgtggggagccggtacgtctgcctttctgtccgtccatgccgatgcgcgttggatcgtctgcgaagtcgccgtagcggacatcatttcgttggaggaaccgggcatggtcaaattcccccgcgctgaagttgttcacgtgggtgatcgcatttcagcttcacacttcatctcggcacgccaagccgatcccgcaagcacgagcaccagcacttcaacttccaccttaacgcctatgccgaccgcgattccgacaccgatgccggccgttgcatcggttactctgccggtcgccgagcaggcccgccatgaagttttcgacgtagcgagtgtttcggccgccgctgcccccgtgaatactctgcctgtcactacgcctcagaaccttcagactgcgacttacgggagcacattatctggggataaccactctcgcttaatcttccgtttgtgggacggtaaacgttatcgccaacttgtggctcgtaccggcgagaacggggttgaggctgacatcccgtactatgtgaacgaggacgacgatattgttgataagccggacgaggacgacgactggatcgaggtgaagggaggaggcagtggcggtggaagtatggattaccccgaggcagtggattggcgcactaagggggcagttactgcggtgaaggatcagggagactgtggtgcatcatacgcatttagcgccatgggtgccttggaaggcgcgaacgccttagcgaaaggcaacgccgtatccctgtcggagcaaaacatcattgattgttcaattccttatggtaaccatggttgtcatggtggaaacatgtatgatgctttcttatacgtcatcgcgaatgagggagtggaccaagattcagcgtatcctttcgtcgggaaacagtcctcttgtaactacaactcgaagtataagggcacgtcgatgagtggtatggtgtctattaagtcgggatcagagtccgatttgcaagcagccgtctctaatgtcggcccagtttcggtggcaattgacggcgcaaactcggccttccgcttttattattctggcgtctatgattccagccgttgttctagttcgtctttgaatcatgcaatggtggtgacggggtacggcagttacaatggaaaaaaatactggcttgccaagaactcctggggcactaactgggggaactcagggtacgttatgatggctcgtaataaatacaatcagtgcggtattgcgacggacgcatcctatcctacgttgtaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcccgggtgatctgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

(19) United States
(12) Patent Application Publication
(10) Pub. No.: US 2025/0283118 A1
(43) Pub. Date: Sep. 11, 2025

(54) MODIFIED BACTERIA AND METHODS OF USE FOR BIOGLASS MICROLENSES

(71) Applicant: University of Rochester, Rochester, NY (US)

(72) Inventors: Anne S. Meyer, Rochester, NY (US); Lynn Sidor, Rochester, NY (US)

(21) Appl. No.: 18/861,915

(22) PCT Filed: May 5, 2023
(86) PCT No.: PCT/US2023/066650

§ 371 Date: Oct. 31, 2024

Related U.S. Application Data
(60) Provisional application No. 63/338,490, filed on May 5, 2022.

(51) Int. Cl. C12P 3/00; C12N 9/64

(52) U.S. Cl. CPC C12P 3/00; C12N 9/641; C07K 2319/00

(57) ABSTRACT

Provided are compositions and methods of generating bioglass microlenses using silicatein expressing cells, as well as compositions comprising the bioglass microlenses and methods of making and using the bioglass microlens compositions.

### CROSS REFERENCE TO RELATED APPLICATIONS

This application claims priority to U.S. Provisional Application No. 63/338,490, filed May 5, 2022 which is hereby incorporated by reference herein in its entirety.

### BACKGROUND OF THE INVENTION

Microlenses are advanced optical devices which can focus light with high efficiency and, as such, have important utilities in imaging, light detection, and light coupling. Generally, microlenses have a diameter on the order of fractions of millimeters and can be used as single lenses for pairing with optical fibers or in arrays for increased efficiency in light collection for applications including charge-coupled devices (CCDs) and photovoltaic cells. Presently several methods exist for fabrication of microlenses, including thermal reflow, microplastic hot embossing, and microdroplet jetting. These methods, however, require labor intensive steps and high energy consumption by expensive specialized equipment and processes.

Increasing technical demands in optical physics have created an urgent need for a new generation of optical devices with novel architectures, increased tunability, and a focus on environmental sustainability. Biological methods of manufacturing bioglasses with unique physical and chemical properties represent a fertile well from which to draw inspiration. Natural materials are typically created through highly ordered processes of spontaneous assembly that act upon simple chemical building blocks (Lakes et al., 1993, Nature, 361 (6412), 511-515; Fratzl et al., 2007, J R Soc Interface, 4 (15), 637-642). Despite their humble origins, biologically fabricated materials display remarkable structural and mechanical properties. Additionally, biological production of materials is highly environmentally friendly, taking place at ambient temperatures and pressures, in aqueous solutions, without utilizing or creating toxic chemicals. Natural optical systems display intriguing hierarchical architecture and a hybrid organic/inorganic composition which can offer outstanding optical properties and suitability for a wide range of potential applications (Aizenberg et al., 2004, J Mater Chem, 14 (14), 2066-2072).

Some aquatic animals are able to grow their own sophisticated optical devices. Glass sponges and brittlestar starfish are both able to control biomineralization reactions, by which they fabricate skeletal elements on both the micro- and macro-scale (Shimizu et al., 1998, Proc Natl Acad Sci USA, 95 (11), 6234-6238; Wiens et al., 2009, Biomaterials, 30 (8), 1648-56; Sundar et al., 2003, Nature, 424 (6951), 899-900). These bio-fabricated optical systems are far more sophisticated than current technology, acting as natural microlens arrays or fiber-optics cables (Sundar et al., 2003, Nature, 424 (6951), 899-900; Aizenberg et al., 2001, Nature, 412 (6849), 819-22). The arms of brittlestar starfish are covered with an array of tightly packed micron-scale hemispherical structures composed of calcite or silica. These microlenses have been called a “nearly perfect optical device” (Aizenberg et al., 2004, J Mater Chem, 14 (14), 2066-2072) due to their light weight, mechanical strength, and lack of aberrations and birefringence (Aizenberg et al., 2001, Nature, 412 (6849), 819-22).

Microlenses are advanced optical components that can collect and focus light with high efficiency. Current techniques for fabricating microlens arrays typically require multiple labor- and energy-intensive steps including baking, developing, and etching (Wu et al., 2002, Adv Mater, 14 (17), 1213-1216; Wu et al., 2002, Langmuir, 18 (24), 9312-9318; Yang et al., 2005, Adv Mater, 17 (4), 435-438; Yang et al., 2005, J Mater Chem, 15 (39), 4200-4202; Yang et al., 2005, Appl Phys Lett, 86 (20), 201121). These approaches are limited to producing microlens arrays only on flat substrates and with comparatively low refractive indices.

Thus, there is a need in the art for an inexpensive and environmentally friendly method for production of bioglass. The present invention addresses this unmet need.

### SUMMARY OF THE INVENTION

In one embodiment, the invention relates to a genetically modified host cell comprising a nucleic acid encoding a silicatein fusion peptide. In one embodiment, the silicatein fusion peptide comprises a fusion of silicatein and at least one selected from the group consisting of a membrane protein and a transmembrane protein domain.

In one embodiment, the cell is a bacterial cell. In one embodiment, the cell is an E. coli cell.

In one embodiment, the silicatein fusion peptide comprises a silicatein sequence of SEQ ID NO:2 or SEQ ID NO:4, or a fragment or variant thereof. In one embodiment, the nucleic acid molecule encoding the silicatein fusion peptide comprises a nucleotide sequence of SEQ ID NO:1 or SEQ ID NO:3, or a fragment or variant thereof.

In one embodiment, the membrane protein is OmpA, OmpC, OmpF, TolC, YaeT or RsaA, or a homolog or ortholog thereof. In one embodiment, the membrane protein comprises a sequence as set forth in SEQ ID NO:6, or a fragment or variant thereof. In one embodiment, the nucleic acid molecule encoding the membrane protein comprises a sequence as set forth in SEQ ID NO:5, or a fragment or variant thereof.

In one embodiment, the transmembrane protein domain is an ice nucleation protein (INP) transmembrane domain. In one embodiment, the INP transmembrane domain comprises a sequence as set forth in SEQ ID NO:8, or a fragment or variant thereof. In one embodiment, the nucleic acid molecule encoding the INP transmembrane domain comprises a sequence as set forth in SEQ ID NO:7.

In one embodiment, the silicatein fusion peptide is SEQ ID NO:14, SEQ ID NO: 17 or SEQ ID NO:20.

In one embodiment, the nucleic acid molecule encoding the silicatein fusion peptide comprises SEQ ID NO: 13, SEQ ID NO: 16 or SEQ ID NO: 19.

In one embodiment, the nucleic acid encoding the silicatein fusion peptide is operatively linked to at least one regulatory element. In one embodiment, the regulatory element is a ribosome binding domain (RBD), a transcriptional termination element, or a double transcriptional termination element. In one embodiment, the regulatory element is an inducible promoter. In one embodiment, the inducible promoter is a lacUV5 promoter or a rhaB promoter.

In one embodiment, the nucleic acid molecule encoding the silicatein fusion peptide comprises a sequence of SEQ ID NO:15, SEQ ID NO: 18 or SEQ ID NO: 21.

In one embodiment, the silicatein fusion peptide is expressed in the presence of at least one inducer molecule specific for induction of the inducible promoter.

In one embodiment, the cell comprises a nucleic acid molecule encoding at least one molecule to alter cellular morphology. In one embodiment, the molecule to alter cellular morphology is BolA, SulA or CrvAB.

In one embodiment, the invention relates to a system for producing a bioglass microlens, the system comprising a cell comprising a nucleic acid encoding a silicatein fusion peptide; and a silicate molecule, or salt, derivative or analog thereof. In one embodiment, the silicate molecule is sodium silicate solution, orthosilicate, tetraethyl orthosilicate (TEOS), tetramethyl orthosilicate, or tetrabutyl orthosilicate.

In one embodiment, the system further comprises an inducer molecule for induction of expression of the silicatein fusion peptide from an inducible promoter. In one embodiment, the inducer molecule is IPTG or rhamnose.

In one embodiment, the invention relates to a method of producing a bioglass microlens, the method comprising contacting a cell comprising a nucleic acid encoding a silicatein fusion peptide with a silicate molecule, or salt, derivative or analog thereof. In one embodiment, the silicate molecule is sodium silicate solution, orthosilicate, tetraethyl orthosilicate (TEOS), tetramethyl orthosilicate, or tetrabutyl orthosilicate.

In one embodiment, the system further comprises contacting the cell with an inducer molecule for induction of expression of the silicatein fusion peptide from an inducible promoter. In one embodiment, the inducer molecule is IPTG or rhamnose.

In one embodiment, the invention relates to a bioglass microlens generated according to the method of producing a bioglass microlens, the method comprising contacting a cell comprising a nucleic acid encoding a silicatein fusion peptide with a silicate molecule, or salt, derivative or analog thereof. In one embodiment, the silicate molecule is sodium silicate solution, orthosilicate, tetraethyl orthosilicate (TEOS), tetramethyl orthosilicate, or tetrabutyl orthosilicate.

In one embodiment, the bioglass microlens comprises a polysilicate layer about 15 nm-25 nm thick, wherein the polysilicate is in a spherical, cylindrical or ovoid formation having a length of about 0.5-3 μm and a radius of about 0.25-1 μm.

In one embodiment, the invention relates to a device comprising at least one bioglass microlens generated according to the method of producing a bioglass microlens, the method comprising contacting a cell comprising a nucleic acid encoding a silicatein fusion peptide with a silicate molecule, or salt, derivative or analog thereof. In one embodiment, the silicate molecule is sodium silicate solution, orthosilicate, tetraethyl orthosilicate (TEOS), tetramethyl orthosilicate, or tetrabutyl orthosilicate. In one embodiment, the device comprises an array of bioglass microlenses.

In one embodiment, the device is an optical device, a solar panel, a solar cell, a compact image sensor, a 3D optical display, a photovoltaic concentrator, a flexible image sensor, a flexible substrate with optical activity, a one-photon microscope, a multiphoton microscope, or a super-resolution emission depletion microscope (STED).

In one embodiment, the invention relates to a method of generating a device comprising at least one bioglass microlens, the method comprising coating a surface or substrate with at least one cell comprising a nucleic acid encoding a silicatein fusion peptide and contacting the coated surface with a silicate molecule, or salt, derivative or analog thereof.

In one embodiment, the silicate molecule is sodium silicate solution, orthosilicate, tetraethyl orthosilicate (TEOS), tetramethyl orthosilicate, or tetrabutyl orthosilicate.

In one embodiment, the method further comprises contacting the coated surface with an inducer molecule for induction of expression of the silicatein fusion peptide from an inducible promoter. In one embodiment, the inducer molecule is IPTG or rhamnose.

In one embodiment, the invention relates to methods of use of a bioglass microlens or a device comprising a bioglass microlens to capture and focus light.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1A through FIG. 1C depict representative imaging of E. coli stained with Rhodamine123. FIG. 1A depicts representative imaging of wildtype Top10 E. coli stained with Rhodamine123. FIG. 1B depicts representative imaging of Top10 E. coli expressing OmpATsil stained with Rhodamine123. FIG. 1C depicts representative imaging of Top10 E. coli expressing OmpASdom stained with Rhodamine 123.

FIG. 2A and FIG. 2B depict representative TEM imaging of E. coli.

FIG. 2A depicts representative TEM imaging of wildtype Top10 E. coli. FIG. 2B depicts representative TEM imaging of Top10 E. coli expressing OmpATsil.

FIG. 3A through FIG. 3C depict representative imaging of E. coli lensing behavior. FIG. 3A depicts an image of representative lensing behavior of wildtype Top10 E. coli. FIG. 3B depicts an image of representative lensing behavior of Top10 E. coli expressing OmpATsil. FIG. 3C depicts an image of representative lensing behavior of Top10 E. coli expressing OmpASdom.

FIG. 4 depicts representative quantification data of the light intensity of E. coli cells after laser illumination.

FIG. 5 depicts representative profiling of light scattered by E. coli cells after laser illumination.

FIG. 6 depicts representative quantification of overall light scattered by E. coli cells after laser illumination.

FIG. 7 depicts representative measurements of the length of light scattered by E. coli cells after laser illumination.

FIG. 8A and FIG. 8B depict induction of a spherical shape in E. coli upon exposure to A22. FIG. 8A depicts representative brightfield microscopy of E. coli expressing OmpATsil when exposed to A22. FIG. 8B depicts representative imaging of Rhodamine123 staining of E. coli expressing OmpATsil when exposed to A22.

FIG. 9A and FIG. 9B depict representative SEM imaging of E. coli.

FIG. 9A depicts representative SEM imaging of wildtype BL21 E. coli. FIG. 9B depicts representative SEM imaging of BL21 E. coli expressing OmpATsil and BolA.

FIG. 10A through FIG. 10E depict representative brightfield microscopy imaging of E. coli elongation upon exposure to cephalexin. FIG. 10A depicts representative imaging of wildtype Top10 E. coli without exposure to cephalexin.

FIG. 10B through FIG. 10E depict representative imaging of wildtype Top10 E. coli after exposure to cephalexin for 15 minutes, 30 minutes, 1 hour, and 2 hours respectively.

FIG. 11A through FIG. 11E depict representative brightfield microscopy imaging of E. coli elongation induced by SulA. FIG. 11A depicts representative imaging of wildtype Top10 E. coli. FIG. 11B through FIG. 11E depict representative imaging of E. coli expressing SulA 15 minutes, 30 minutes, 1 hour, and 2 hours after induction, respectively.

FIG. 12 depicts the results of an alamarBlue assay which was performed on wild-type E. coli cells, as well as E. coli cells expressing OmpA-silicatein constructs.

FIG. 13 depicts the results of a colony-forming unit (CFU) experiment which was performed on wild-type E. coli cells, as well as E. coli cells expressing OmpA-silicatein constructs, where the silicatein enzyme was derived either from T. aurantia or S. domuncula.

### DETAILED DESCRIPTION OF THE INVENTION

The invention is based, in part, on the development of engineered Escherichia bacteria which have been engineered to express a silicatein enzyme with the ability to mineralize monomeric silica into polysilicate bioglass that is fused to an E. coli outer membrane protein. When this protein is expressed, the bacteria produces and displays the sponge silicatein enzyme on its outer surface. Upon incubation with silicate, the displayed silicatein enzymes mineralize a bioglass coating around the outside of the cells. The self-assembled, bioglass-coated cells can then capture and focus light, thereby behaving like microlenses.

In some embodiments, the invention provides engineered Escherichia bacteria which have been engineered to express a silicatein enzyme on the outer surface. In some embodiments, the engineered Escherichia bacteria express a silicatein fusion protein.

In some embodiments, the invention provides systems and methods for generating bioglass-coated cells and bioglass microlenses by contacting the engineered Escherichia bacteria which have been engineered to express a silicatein enzyme on the outer surface with silicate.

In some embodiments, the invention provides systems and methods for generating compositions comprising bioglass microlenses and methods of use thereof to capture and focus light.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

“Antisense,” as used herein, refers to a nucleic acid sequence which is complementary to a target sequence, such as, by way of example, complementary to a target miRNA sequence, including, but not limited to, a mature target miRNA sequence, or a sub-sequence thereof. Typically, an antisense sequence is fully complementary to the target sequence across the full length of the antisense nucleic acid sequence.

“Complementary” as used herein refers to the broad concept of subunit sequence complementarity between two nucleic acids. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, at least about 60% or at least about 80% of corresponding positions in each of the molecules are occupied by nucleotides which normally base pair with each other (e.g., A:T and G:C nucleotide pairs).

An “enhancer” is a nucleic acid sequence that acts to potentiate the transcription of a transcriptional unit independent of the identity of the transcriptional unit, the position of the sequence in relation to the transcriptional unit, or the orientation of the sequence. The vectors of the present invention optionally include enhancers.

The term “expression” as used herein is defined as the transcription and/or translation of a particular nucleotide sequence.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, and plasmids (e.g., naked or contained in liposomes).

“Fragment” may mean a polypeptide fragment of an protein that is biological property as the full length protein. A fragment of an protein may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length protein, excluding any heterologous signal peptide added. The fragment may comprise a fragment of a polypeptide that is 95% or more, 96% or more, 97% or more, 98% or more or 99% or more identical to the protein and additionally comprise an N terminal methionine or heterologous signal peptide which is not included when calculating percent identity. Fragments may further comprise an N terminal methionine. The N terminal methionine may be linked to a fragment of an protein. A fragment of a nucleic acid sequence that encodes an protein may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the protein and additionally optionally comprise sequence encoding an N terminal methionine which is not included when calculating percent identity. Fragments may further comprise coding sequences for an N terminal methionine. The coding sequence encoding the N terminal methionine may be linked to a fragment of coding sequence.

“Heterologous” or “Heterologous sequence” means a protein sequence or nucleic acid sequence that is foreign to the host (e.g., a nucleic acid sequence originating from a donor that is different than the host, or a chemically synthesized nucleic acid sequence that encodes an expression product such as a polypeptide that is foreign to the host). In the case where the host is a particular prokaryotic species, the heterologous nucleic acid sequence may have originated from a different species within the same genus, or from a different genus or family, or from a different order or class, or from a different phylum (division), or from a different domain (empire) of organisms. The heterologous nucleic acid sequence originating from a donor different from the host can be modified, before it is introduced into a host cell, by making mutations, insertions, deletions or substitutions in the nucleic acid seqeucne as long as such modified sequences exhibit the same or similar function (functionally equivalent) as the starting donor sequence. A heterologous nucleic acid sequence, as referred to herein, encompasses nucleic sequences originating from a different domain of organisms. For example, heterologous nucleic acid sequencef may originate from eukaryotes. Heterologous nucleic acids may also include nucleic acids or a part of the nucleic acid sequences have been modified according to the codon usage of a host.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology.

As used herein, “homology” is used synonymously with “identity.”

The terms “host,” “host cell,” “genetically engineered host,” and “genetically engineered host cell” are used interchangeably herein to indicate a prokaryotic or eukaryotic cell into which one or more vectors, or isolated and purified nucleic acid sequences, of the invention have been introduced. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Measuring” or “measurement,” or alternatively “detecting” or “detection,” means assessing the presence, absence, quantity or amount (which can be an effective amount) of either a given substance within a sample, including the derivation of qualitative or quantitative concentration levels of such substances, or otherwise evaluating the values or categorization of the substance or the sample.

“Naturally occurring” as used herein describes a composition that can be found in nature as distinct from being artificially produced. For example, a nucleotide sequence present in an organism, which can be isolated from a source in nature and which has not been intentionally modified by a person, is naturally occurring.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Operon” means one or more loci operably linked to a regulatory region such that, under appropriate conditions, an RNA polymerase may bind to a promoter sequence in the regulatory region and proceed to transcribe the loci. The loci within an operon share a common regulatory region and, therefore, are substantially regulated as a unit. Amongst the loci in an operon may be a repressor locus which encodes a repressor protein which, under appropriate conditions, binds to the operator of the operon so as to substantially decrease expression of the loci in the operon.

A nucleic acid sequence is “operably linked” or “operatively linked” when it is placed into a functional relationship with another nucleic acid sequence. For example, a promoter is operably linked to a coding sequence if it affects the transcription of the sequence; or a translation initiation region such as a ribosome binding site is operably linked to a nucleic acid sequence encoding, for example, a polypeptide if it is positioned so as to facilitate translation of the polypeptide. Linking can be accomplished by ligation at convenient restriction sites. If such sites do not exist, the synthetic oligonucleotide adaptors or linkers are used in accordance with conventional practice.

As used herein, “polynucleotide” includes cDNA, RNA, DNA/RNA hybrid, anti-sense RNA, siRNA, miRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences.

The term “promoter” as used herein is defined as a DNA sequence recognized by the synthetic machinery of the cell, or introduced synthetic machinery, required to initiate the specific transcription of a polynucleotide sequence. As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, express the gene product in a temporally specific manner, a spatially specific manner or be constitutive.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be substantially produced in a cell only when an inducer which corresponds to the inducible promoter is present in the cell.

The term “recombinant DNA” as used herein is defined as DNA produced by joining pieces of DNA from different sources.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Transcriptional unit” as used herein refers to a nucleic acid sequence that is normally transcribed into a single RNA molecule. The transcriptional unit might contain one gene (monocistronic) or two (dicistronic) or more genes (polycistronic) that code for functionally related polypeptide molecules.

“Transformation” refers to the introduction of a plasmid into an organism so that the plasmid is replicable, either as an extrachromosomal element or by chromosomal integration. Methods of transforming bacterial and eukaryotic hosts are well known in the art, many of which methods are summarized in Sambrook, et al., Molecular Cloning: A Laboratory Manual, (2012). Successful transformation is generally recognized when any indication of the operation of this plasmid occurs within the host cell. For example, a sensitive host cell will become resistant to a selecting agent when the host cell is transfected with a plasmid that allows for the resistance.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides compositions, methods, systems and cells for generating micobial biolenses. In some embodiments, the systems and compositions of the invention comprise cells that express a silicatein fusion peptide on their membrane surface which forms a polysilicate coating (e.g, bioglass) in the presence of silicate. In one embodiment, the composition comprises a bioglass-coated cell. For example, in various embodiments, the bio-glass coated cells can be used to as polysilicate particles with a range of powerful optical properties, with control at the micro- and nano-scale.

In one aspect, the invention provides a genetically engineered cell. In one embodiment, the genetically engineered cell is a genetically engineered host cell. In one embodiment, the genetically engineered cell comprises a nucleic acid encoding a silicatein fusion peptide for expression on the surface of the cell. In one embodiment, the genetically engineered cell comprises a nucleic acid encoding a silicatein fusion peptide comprising silicatein operably linked to an outer membrane protein or a transmembrane domain.

In some embodiments, the genetically engineered cell of the invention comprises an altered cellular morphology. Therefore, in some embodiments, the genetically engineered cell of the invention further comprises an isolated nucleic acid comprising a molecule for altering cellular morphology. Exemplary molecule for altering cellular morphology include, but are not limited to, BolA, SulA, and CrvAB. In some embodiments, the genetically engineered cell of the invention is contacted with or cultured in the presence of a molecule for altering cell morphology. In some embodiments, the molecule for altering cell morphology is S-(3,4-dichlorobenzyl) isothiourea (A22).

The genetically engineered host cell can be any type of cell suitable for protein expression. For example, in some embodiments, the genetically engineered host cell is a prokaryotic cell, including a bacterial cell. Exemplary bacterial cells include, but are not limited to, E. coli, Streptomyces, Rhodobacter, Salmonella, Shigella, Bacillus, Streptococcus, Lactobacillus, Caulobacter, Rhodococus, Pseudomonas, Aeromonas, Clostridium or Haemophilus Influenza. Alternatively, the cell may be a eukaryotic cell, including a yeast cell. Exemplary yeast cells include, but are not limited to Saccharomyces cerevisiae.

In another aspect, the invention provides a system for producing a bio-glass coated cell. In some embodiments, the system comprises a genetically engineered cell of the invention comprising a nucleic acid encoding a silicatein fusion peptide for expression on the surface of the cell under the control of an inducible promoter, an inducer molecule for induction of expression of the silicatein fusion peptide and a silicate molecule, or analog, salt or derivative thereof to promote the formation of polysilicate on the surface of the cell. In some embodiments, the silicate molecule is orthosilicate, or analog, salt or derivative thereof. In some embodiments, the system further comprises an agent to alter the morphology of the cell.

In one embodiment, the inducible promoter comprises a rhaB promoter and the inducible molecule comprises rhamnose. In one embodiment, the inducible promoter comprises a lacUV5 promoter and the inducible molecule comprises IPTG.

In one embodiment, the method of the invention comprises providing a genetically engineered cell of the invention, incubating the cell in a media; and adding an inducer molecule for induction of expression of the silicatein fusion peptide to the media; and adding a silicate molecule, or analog, salt or derivative thereof to promote the formation of polysilicate on the surface of the cell. In some embodiments, the silicate molecule is orthosilicate, or analog, salt or derivative thereof. In some embodiments, the method further comprises contacting the cell with an agent to alter the morphology of the cell.

In one embodiment, the present invention provides devices and systems comprising the polysilicate coated cells, or bioglass microlenses, of the invention, and methods of use thereof. Exemplary devices that can incorporate the polysilicate coated cells, or bioglass microlenses, of the invention include, but are not limited to, solar cells, compact image sensors, 3D displays, concentrators for photovoltaics, flexible image sensors, flexible substrates with optical activity, one-photon and multiphoton microscopy, super-resolution emission depletion microscopy (STED), and optogenetic stimulation and readouts.

### Silicatein Expressing Cells

In one aspect, the present invention provides a cell and compositions comprising a cell expressing a fusion peptide of silicatein. In some embodiments, the cell is a genetically engineered host cell engineered to express a silicatein fusion peptide of the invention. In one embodiment, the fusion peptide comprises an outer membrane protein (OMP)-silicatein fusion peptide. In one embodiment, the outer membrane protein comprises E. coli OmpA, OmpC, OmpF, TolC, and YaeT, or a homolog or ortholog thereof. In one embodiment, the outer membrane protein comprises Caulobacter crescentus RsaA, or a homolog or ortholog thereof. In one embodiment, the fusion peptide comprises a protein transmembrane domain-silicatein fusion peptide. In one embodiment, the fusion peptide comprises an ice nucleation protein (INP) transmembrane domain-silicatein fusion peptide. In some embodiments, expression of the silicatein fusion peptide of the invention is under the control of an inducible promoter.

In one embodiment, the cell comprises a nucleic acid encoding a silicatein fusion peptide. In one embodiment, the nucleic acid encoding the silicatein fusion peptide is integrated into the host cell genome. In one embodiment, the nucleic acid encoding the silicatein fusion peptide is exogenous to the host cell genome.

In one embodiment, the silicatein is from a marine sponge. Exemplary marine sponge silicatein enzymes that can be used include, but are not limited to, Suberites domuncula silicatein, Tethya aurantium silicatein, Lubomirskia baicalensis silicatein, Euplectella aspergillum silicatein, Aulosaccus AuSil-Hexa silicatein, Ephydatia fluviatils silicatein, Hymeniacidon perlevis silicatein, and Petrosia ficiformis silicatein.

In one embodiment, the nucleic acid encoding a silicatein fusion peptide encodes a silicatein sequence as set forth in SEQ ID NO:2 or SEQ ID NO:4, or a fragment or variant thereof, operably linked to a sequence encoding an outer membrane protein or a transmembrane protein domain. In one embodiment, the nucleic acid encoding a silicatein fusion peptide comprises a sequence as set forth in SEQ ID NO: 1 or SEQ ID NO:3, or a fragment or variant thereof, operably linked to a nucleotide sequence encoding an outer membrane protein or a transmembrane protein domain. In some embodiments, the silicatein amino acid sequence is fused to the C-terminus of the outer membrane protein or transmembrane protein domain. In some embodiments, the silicatein amino acid sequence is fused to the N-terminus of the outer membrane protein or transmembrane protein domain. In some embodiments, the fusion peptide of the invention comprises a linker sequence between the silicatein sequence and the outer membrane protein or transmembrane protein domain.

In one embodiment, the nucleic acid encoding a silicatein fusion peptide encodes a silicatein sequence as set forth in SEQ ID NO: 2 or SEQ ID NO:4, or a fragment or variant thereof, operably linked to an OMP comprising a sequence as set forth in SEQ ID NO:6, or a fragment or variant thereof. In one embodiment, the silicatein fusion peptide can comprise an amino acid sequence of SEQ ID NO:14 or SEQ ID NO: 17, or a fragment or variant thereof. In one embodiment, the nucleic acid encoding a silicatein fusion peptide comprises a sequence as set forth in SEQ ID NO: 1 or SEQ ID NO: 3, or a fragment or variant thereof, operably linked to a sequence as set forth in SEQ ID NO: 11, or a fragment or variant thereof. In one embodiment, the nucleic acid molecule comprises a nucleotide sequence comprising SEQ ID NO:13 or SEQ ID NO:16, or a fragment or variant thereof. In one embodiment, the nucleic acid molecule comprises a nucleotide sequence as set forth in SEQ ID NO:15 or SEQ ID NO:18, or a fragment or variant thereof.

In one embodiment, the nucleic acid encoding a silicatein fusion peptide encodes a silicatein sequence as set forth in SEQ ID NO:2 or SEQ ID NO:4, or a fragment or variant thereof, operably linked to an INP transmembrane domain comprising a sequence as set forth in SEQ ID NO:8, or a fragment or variant thereof. In one embodiment, the silicatein fusion peptide can comprise an amino acid sequence of SEQ ID NO:20, or a fragment thereof or variant thereof. In one embodiment, the nucleic acid encoding a silicatein fusion peptide comprises a sequence as set forth in SEQ ID NO: 1 or SEQ ID NO:3, or a fragment or variant thereof, operably linked to a sequence as set forth in SEQ ID NO:7, or a fragment or variant thereof. In one embodiment, the nucleic acid molecule comprises a nucleotide sequence comprising SEQ ID NO:19, or a fragment thereof or variant thereof. In one embodiment, the nucleic acid molecule comprises a nucleotide sequence as set forth in SEQ ID NO:21, or a fragment thereof or variant thereof.

The present invention should also be construed to encompass “variants,” and “fragments” of the silicatein fusion peptide sequences described herein (or of the DNA encoding the same) which variants are altered in one or more amino acids (or, when referring to the nucleotide sequence encoding the same, are altered in one or more base pairs) such that the resulting peptide (or DNA) is not identical to the sequences recited herein, but has the same biological property as the polypeptides disclosed herein, in that the peptide has biological/biochemical properties. A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

Design, generation, and testing of the variant nucleic acid sequences, and/or their encoded polypeptides, having the above required percent identities to the silicatein fusion peptides as described herein and retaining the required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 80-99% identity to the silicatein fusion peptide sequences described herein and screen such for activity according to methods routine in the art. Generally, conservative substitutions can be made at any position so long as the required activity is retained.

A polynucleotide encoding a silicatein fusion peptide, as described above, can be inserted into a bacterial expression vector by a variety of means known to the art. For example, design and construction of a strain can be achieved through complementary end PCR product ligation cloning and homologous recombination. Other suitable methods will be known to those skilled in the art.

The genetically engineered host cell can further comprise a nonintegrated expression vector encoding the silicatein fusion peptide, wherein the expression vector comprises the silicatein fusion peptide encoding sequence under control of a promoter. In one embodiment, the promoter is recognized by the heterologous RNA polymerase. In one embodiment, the promoter is a T7 promoter, a T7lac promoter (containing lacO operator sites), T7 promoter region, a TI promoter, or variants thereof. In one embodiment, the nucleic acid encoding the silicatein fusion peptide is under the control of an inducible promoter or a variant thereof. Accordingly, the silicatein fusion peptide is expressed in the presence of an inducer molecule for induction of the inducible promoter or alternatively repressed in the presence of an inhibitory molecule for repression of the inducible promoter. Exemplary inducible promoter and inducers include, but are not limited to, the rhamnose-regulated (rhaPBAD) promoter which is regulated by two activators, RhaS and RhaR, a negative inducible lac promoter, or a fragment or variant thereof, which regulated by the LacI repressor and can be induced with IPTG (e.g., lacP1, lacUV5, Alul lac, HaelII lac fragment, lacPs, etc.), TetON promoters which are inducible in the presence of Tetracycline or a derivative thereof, promoter pBad which is repressed by AraC in the absence of arabinose and induced in the presence of high arabinose and low glucose, a light switchable promotor, or other promoters known to those of skill in the art which can be used to temporally control the expression of the silicatein fusion peptide.

The present invention can be used with a variety of suitable prokaryotic hosts. In some embodiments, the prokaryotic host is E. coli, Streptomyces, Rhodobacter, Salmonella, Shigella, Bacillus, Lactobacillus, Caulobacter, Rhodococus, Pseudomonas, Aeromonas, Clostridium, or Haemophilus Influenza. For example, suitable host E. coli strains include, but are not limited to, Top10, BL21, C2566, DH1, DH41, DH5, DH51, DH5IF′, DH5IMCR, DH10B, DH10B/p3, DH11S, C600, HB101, JM101, JM105, JM109, JM110, K12, K38, MG1655, RR1, Y1088, Y1089, CSH18, ER1451, and ER1647. Other suitable hosts are known in the art (see e.g., Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253). In one embodiment, the host is a Top10 E. coli strain.

Suitable Bacilli host strains include, but are not limited to lactobacillus, B. subtilis, B. licheniformis, B. coagulans, B. megaterium, B. amyloliquefaciens, B. halodurans, B. cereus, bacteroides and alkalophilic bacilli.

The present invention can be used with a variety of suitable eukaryotic hosts. In some embodiments, the eukaryotic host is a yeast host. For example, suitable eukaryotic hosts include, but are not limited to, S. cerevisiae, Pichia, Kluyveromyces, Schizosaccharomyces and Hansenula.

Genetically engineered host cells developed according to the approaches described herein can be evaluated by a number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253). For example, host cells can be conveniently tested for degree of control, leakiness of expression, and induction capacity using method known to those of skill in the art. As an example, a detectable protein such as green fluorescence protein (or its derivatives) or luciferase can be used as an indicator. An advantage of using these reporter genes is that no protein purification and concentration is necessary, and it can be assayed very sensitively.

Host cells are usually cultured in conventional media as known in the art. For example, suitable growth media are common commercially prepared media such as Bacto Lactobacilli MRS broth or Agar (Difco), Luria Bertani (LB) broth, Terrific broth (TB), Sabouraud Dextrose (SD) broth or Yeast Medium (YM) broth. Other defined or synthetic growth media, such as M9 media for E. coli, may also be used, and the appropriate medium for growth of the particular bacterial strain will be known by one skilled in the art of microbiology or fermentation science. The medium might be modified as appropriate, for example, by adding further ingredients such as buffers, salts, vitamins, cofactors or amino acids. Different media or combinations of media can be used during the culturing of the cells.

In some embodiments, the genetically engineered cell of the invention further comprises an isolated nucleic acid comprising a transcriptional regulator. Exemplary transcriptional regulators include, but are not limited to, BolA.

As cell culture systems, continuous or discontinuous culture such as batch culture or fed batch culture can be applied in culture tubes, shake flasks or bacterial fermenters.

In some embodiments, the genetically engineered cell of the invention comprises an altered the cell morphology as compared to a standard or wild-type rod-shaped bacterial cell. In some embodiments, the engineered cell of the invention comprises a spherical shape, a filamentous shape, a crescent shape, a helical shape, a snake-like shape, or a lemon shape or another cellular morphology. In some embodiments the genetically engineered cell of the invention comprises a molecule for altering cellular morphology. Exemplary molecules for altering the cell shape include, but are not limited to, BolA, inhibitors of FtsZ, activators of SulA protein expression or activity, activators of CrvAB protein expression or activity, activators of MinC/D protein expression or activity, S-layer proteins from Lysinibacillus sphaericus JG-A12, inhibitors of Lon function, activators of the bacterial SOS response, inhibitors of MreB, or other factors that can alter the morphology of the bacterial cell. In some embodiments, the genetically engineered cell of the invention comprises one or more mutations that alter the morphology of the bacterial cell. Exemplary mutations that can be introduced into the genetically altered cell of the invention that can alter cellular morphology and promote the formation of cells with an altered cellular morphology include, but are not limited to PBP2 mutations, ftsZ mutations, and metK mutations.

In some embodiments, the genetically engineered cell of the invention is contacted with or cultured in the presence of one or more agents that alters the morphology of the bacterial cell. Exemplary agents that can be used to alter the morphology of the bacterial cell include, but are not limited to, small molecule inhibitors of FtsZ (e.g., Zantrins, viriditoxin), inhibitors of MreB (e.g., S-(3,4-dichlorobenzyl) isothiourea (A22)), cephalexin or other small molecule inhibitors that can alter cellular morphology.

### Nucleic Acids and Vectors

In one aspect, the invention provides nucleic acids encoding a heterologous protein to be expressed. In one embodiment, the nucleic acid comprises a promotor and a sequence encoding a protein operatively linked to the promoter. In some embodiments, the nucleic acid can be incorporated into a vector for delivery to a cell.

In brief summary, the expression of natural or synthetic nucleic acids encoding a polypeptide or protein will typically be achieved by operably linking a nucleic acid encoding the heterologous protein or portions thereof to a promoter, a ribosome binding site (RBS), and one or more transcriptional terminators and incorporating the construct into an expression vector. In some embodiments, the promoter and RBS are positioned upstream or 5′ to the sequence encoding the fusion peptide and one to more transcriptional terminators are positioned downstream or 3′ to the sequence encoding the fusion peptide. An exemplary RBS that can be included in the expression vectors of the invention includes, but is not limited to, SEQ ID NO:11. An exemplary transcriptional termination sequence that can be included in the expression vectors of the invention includes, but is not limited to, SEQ ID NO:12.

The vectors can be suitable for replication and integration. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, and a cosmid. Vectors of particular interest include expression vectors, replication vectors, probe generation vectors, and sequencing vectors. In some aspects, the expression vector includes, but is not limited to a plasmid, a bacterial vector. Numerous expression vector systems exist that comprise at least a part or all of the compositions discussed above. Prokaryote- and/or eukaryote-vector based systems can be employed to produce polynucleotides, or their cognate polypeptides.

Further, the expression vector may be provided to a cell of the invention, as described elsewhere herein, in the form of a plasmid. The plasmids may be introduced into the cells by transformation or transfection. There are multiple methods for transfecting agents into a cell including, but not limited to, electroporation, chemical transformation, calcium phosphate-based transfections, DEAE-dextran-based transfections, lipid-based transfections, molecular conjugate-based transfections (e.g., polylysine-DNA conjugates), microinjection and others. In some embodiments, the vector contains one or more of an origin of replication functional in at least one organism, a promoter sequence, convenient restriction endonuclease sites, and one or more selectable markers. (e.g., WO 01/96584; WO 01/29058; and U.S. Pat. No. 6,326,193).

For expression of the polypeptides of the invention or portions thereof, at least one module in each promoter functions to position the start site for RNA synthesis. The best known example of this is the TATA box (or Pribnow box in prokaryotes), but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, such as for example, enhancers and UP DNA sequences, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, individual elements can function either co-operatively or independently to activate transcription.

In one embodiment, the coding nucleic acid sequence encoding a heterologous protein is under the control of a recombinant or heterologous promoter, which refers to a promoter that is not normally associated with a polynucleotide sequence in its natural environment. An enhancer may be one naturally associated with a polynucleotide sequence, located either downstream or upstream of that sequence. Such an enhancer can be referred to as “endogenous.” Alternatively, a recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (e.g., U.S. Pat. Nos. 4,683,202, 5,928,906).

Naturally, it will be important to employ a promoter and/or enhancer that effectively directs the expression of the DNA segment in the cell type, organelle, and organism chosen for expression.

In one embodiment, the promoter has been modified to include the lactose operator (lacO). The lacO is a binding site for the lactose operon repressor. The lactose repressor binds the lacO, which prevents the RNA polymerase from binding the lac promoter, thus effectively repressing expression of the target protein. The repression is reversible upon addition of an inducing agent to the host cell. The inducing agent knocks the lactose repressor off the lacO and allows the RNA polymerase to bind the lac promoter and initiate expression of the target protein. Inclusion of lacO tightens the initiation of expression of the target protein by nearly 10-fold. This also helps to reduce the potential for expression of the target protein prior to induction, which for some target proteins, have deleterious effects on host cell growth, thus, reducing maximum target protein production. In one embodiment, the lactose repressor is produced from an endogenous host cell gene called lacI. Alternatively, host cell may also contain one or more heterologous lacI-genes.

Full expression of a target gene from an expression clone with a lac promoter requires release of the lac repressor from its binding site in the lac promoter. This event can be triggered by release of the lac repressor by, for example, addition of IPTG or by the presence of lactose in the medium.

In one embodiment, the promoter is a lacUV5 promoter or a variant thereof. In one embodiment the lacUV5 promoter comprises a sequence of SEQ ID NO: 9.

In some embodiments, the promoter may be responsive to the level of arabinose in the environment. Generally speaking, arabinose may be present during the in vitro growth of a bacterium, while typically absent from host tissue. In one embodiment, the promoter is derived from an araC-P_{BAD} system. The araC-P_{BAD} system is a tightly regulated expression system, which has been shown to work as a strong promoter induced by the addition of low levels of arabinose. The araC-araBAD promoter is a bidirectional promoter controlling expression of the araBAD nucleic acid sequences in one direction, and the araC nucleic acid sequence in the other direction. For convenience, the portion of the araC-araBAD promoter that mediates expression of the araBAD nucleic acid sequences, and which is controlled by the araC nucleic acid sequence product, is referred to herein as P_{BAD}:). For use as described herein, a cassette with the araC nucleic acid sequence and the araC-araBAD promoter may be used. This cassette is referred to herein as araC-P_{BAD}. The AraC protein is both a positive and negative regulator of P_{BAD}. In the presence of arabinose, the AraC protein is a positive regulatory element that allows expression from P_{BAD}. In the absence of arabinose, the AraC protein represses expression from P_{BAD}. This can lead to a 1,200-fold difference in the level of expression from P_{BAD}.

Other enteric bacteria contain arabinose regulatory systems homologous to the araC-araBAD system from E. coli. For example, there is homology at the amino acid sequence level between the E. coli and the S. Typhimurium AraC proteins, and less homology at the DNA level. However, there is high specificity in the activity of the AraC proteins. For example, the E. coli AraC protein activates only E. coli P_{BAD} (in the presence of arabinose) and not S. Typhimurium P_{BAD}. Thus, an arabinose regulated promoter may be used in a recombinant bacterium that possesses a similar arabinose operon, without substantial interference between the two, if the promoter and the operon are derived from two different species of bacteria.

Generally speaking, the concentration of arabinose necessary to induce expression is typically less than about 2%. In some embodiments, the concentration is less than about 1.5%, 1%, 0.5%, 0.2%, 0.1%, or 0.05%. In other embodiments, the concentration is 0.05% or below, e.g. about 0.04%, 0.03%, 0.02%, or 0.01%. In an exemplary embodiment, the concentration is about 0.05%. In some embodiments, the concentration of arabinose used for induction of expression is between 10 mM and 50 mM.

In other embodiments, the promoter may be responsive to the level of maltose in the environment. Generally speaking, maltose may be present during the in vitro growth of a bacterium, while typically absent from host tissue. The malT nucleic acid sequence encodes MalT, a positive regulator of four maltose-responsive promoters (P_{PQ}, P_{EFG}, P_{KBM}, and P_{S}). The combination of malT and a mal promoter creates a tightly regulated expression system that has been shown to work as a strong promoter induced by the addition of maltose. Unlike the araC-P_{BAD} system, malT is expressed from a promoter (P_{T}) functionally unconnected to the other mal promoters. P_{T} is not regulated by MalT. The malEFG-malKBM promoter is a bidirectional promoter controlling expression of the malKBM nucleic acid sequences in one direction, and the malBFG nucleic acid sequences in the other direction. For convenience, the portion of the malEFG-malKBM promoter that mediates expression of the malKBM nucleic acid sequence, and which is controlled by the malT nucleic acid sequence product, is referred to herein as P_{KBM}, and the portion of the malEFG-malKBM promoter that mediates expression of the malEFG nucleic acid sequence, and that is controlled by the mall nucleic acid sequence product, is referred to herein as P_{EFG}. Full induction of P_{KBM} requires the presence of the MalT binding sites of P_{EFG}. For use in the vectors and systems described herein, a cassette with the mall nucleic acid sequence and one of the mal promoters may be used. This cassette is referred to herein as ma/T-Pₘₐₗ. In the presence of maltose, the MalT protein is a positive regulatory element that allows expression from Pₘₐₗ.

In still other embodiments, the promoter may be sensitive to the level of rhamnose in the environment. Analogous to the araC-P_{BAD} System described above, the rhaRS-P_{rhaB} activator-promoter system is tightly regulated by rhamnose. Expression from the rhamnose promoter (Pᵣₕₐ) is induced to high levels by the addition of rhamnose, which is common in bacteria but rarely found in host tissues. The nucleic acid sequences rhaBAD are organized in one operon that is controlled by the P_{rhaBAD} promoter (or rhaB). This promoter is regulated by two activators, RhaS and RhaR, and the corresponding nucleic acid sequences belong to one transcription unit that is located in the opposite direction of the rhaBAD nucleic acid sequences. If L-rhamnose is available, RhaR binds to the P_{rhaRS} promoter and activates the production of RhaR and RhaS. RhaS together with L-rhamnose in turn binds to the P_{rhaBAD} and the P_{rhaT} promoter and activates the transcription of the structural nucleic acid sequences. Full induction of rhaBAD transcription also requires binding of the Crp-cAMP complex, which is a key regulator of catabolite repression.

Although both L-arabinose and L-rhamnose act directly as inducers for expression of regulons for their catabolism, important differences exist in regard to the regulatory mechanisms. L-Arabinose acts as an inducer with the activator AraC in the positive control of the arabinose regulon. However, the L-rhamnose regulon is subject to a regulatory cascade; it is therefore subject to even tighter control than the araC P_{BAD} System. L-Rhamnose acts as an inducer with the activator RhaR for synthesis of RhaS, which in turn acts as an activator in the positive control of the rhamnose regulon. In the present invention, rhamnose may be used to interact with the RhaR protein and then the RhaS protein may activate transcription of a nucleic acid sequence operably-linked to the P_{rhaBAD} promoter.

Generally speaking, the concentration of rhamnose necessary to induce expression is typically less than about 1%. In some embodiments, the concentration is less than about 0.5%, 0.2%, 0.1%, or 0.05%. In other embodiments, the concentration is 0.05% or below, e.g. about 0.04%, 0.03%, 0.02%, or 0.01%. In an exemplary embodiment, the concentration is about 0.2%.

In one embodiment, the promoter is a P_{rhaBAD} promoter or a variant thereof. In one embodiment the P_{rhaBAD} promoter comprises a sequence of SEQ ID NO: 10.

In still other embodiments, the promoter may be sensitive to the level of xylose in the environment. The xyIR-P_{xyla}, system is another well-established inducible activator-promoter system. Xylose induces xylose-specific operons (xylE, xylFGHR, and xylAB) regulated by XylR and the cyclic AMP-Crp system. The XylR protein serves as a positive regulator by binding to two distinct regions of the xyl nucleic acid sequence promoters. As with the araC-P_{BAD} System described above, the xylR-P_{xylAB} and/or xy/R—P_{xylFGH} regulatory systems may be used in the present invention. In these embodiments, xylR P_{xylAB} Xylose interacting with the XylR protein activates transcription of nucleic acid sequences operably-linked to either of the two Pay promoters.

The nucleic acid sequences of the promoters detailed herein are known in the art, and methods of operably-linking them to a chromosomally integrated nucleic acid sequence encoding a repressor are known in the art and detailed in the examples.

In order to assess the expression of the polypeptides of the invention or portions thereof, the expression vector to be introduced into a cell of the invention can also contain either a selectable marker gene or a reporter gene or both to facilitate identification and selection of expressing cells from the population of cells sought to be transfected, transformed or infected through vectors, plasmid, viral or phage, for example. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, but are not limited to, antibiotic-resistance genes, such as zeocin (Sh ble gene), neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity or fluorescence. Expression of the reporter gene is assayed at a suitable time after the DNA has been introduced into the recipient cells.

Suitable reporter genes may include, but are not limited to, genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the invention includes a tag polypeptide that can be covalently linked thereto to the polypeptides of the invention. That is, the invention encompasses a recombinant nucleic acid wherein the nucleic acid encoding the tag polypeptide is covalently linked to the nucleic acid of the polypeptides of the invention. Such tag polypeptides are well known in the art and include, for instance, green fluorescent protein (GFP), myc, myc-pyruvate kinase (myc-PK), His₆, maltose binding protein (MBP), thiol disulfide oxidoreductases (DsbA), outer membrane proteins or domains (such as OmpA) an influenza virus hemagglutinin tag polypeptide, a flag tag polypeptide (FLAG), a glutathione-S-transferase (GST) tag polypeptide, and a EGFP protein. However, the invention should in no way be construed to be limited to the nucleic acids encoding the above-listed tag polypeptides. Rather, any nucleic acid sequence encoding a polypeptide which may function in a manner substantially similar to these tag polypeptides should be construed to be included in the present invention. Further, addition of a tag polypeptide facilitates isolation and purification of the “tagged” protein such that the protein of the invention can be produced and purified readily.

The vectors of the invention optionally may include one or more marker sequences. Generally speaking, suitable marker sequences typically encode a gene product, usually an enzyme that inactivates or otherwise detects or is detected by a compound in the growth medium. For example, the inclusion of a marker sequence may render the transformed cell resistant to an antibiotic, or it may confer compound-specific metabolism on the transformed cell. Examples of a suitable marker sequences that confer resistance include, but are not limited to, kanamycin, ampicillin, chloramphenicol, zeocin, spectinomycin and tetracycline. Alternatively, rather than selective pressure, a marker gene may be used that allows for detection of particular colonies containing the gene, such as beta-galactosidase, where a substrate is employed that provides for a colored product.

Useful vectors include, but are not limited to, pD431, PD434, pD451, pD454 (DNA 2.0 (now ATUM)), pET3 (Novagen), pRSET (Thermo-Fisher), pBluescript vectors, Phagescript vectors, pNH8A, [rho]NH16a, pNH18A, [rho]NH46A (Stratagene Cloning Systems, Inc.); ptrc99a, pKK223-3, [rho]KK233-3, pDR540, pRIT5 (Pharmacia Bio-tech, Inc.); pLG338, pACYC184, pBR322, pUC18, pUC19, pKC30, pRep4, pACYC177, pACYC184, pRSFIOIO and pBW22 (Wilms et al., 2001, Biotechnology and Bioengineering, 73 (2) 95-103) or variants thereof. Further useful plasmids are well known to the person skilled in the art and are described e.g., in “Cloning Vectors” (Eds. Pouwels P. H. et al. Elsevier, Amsterdam-New York-Oxford, 1985).

### Expression Systems

In one aspect, the invention provides a system for expressing a silicatein fusion peptide. In one embodiment, the system comprises a genetically engineered host cell engineered to express a heterologous silicatein fusion peptide. In one embodiment, the genetically engineered host cell comprises a nucleic acid sequence encoding the heterologous silicatein fusion peptide integrated into the host cell genome. Alternatively, the heterologous RNA polymerase can be inserted into the host genome as an exogenous construct. In one embodiment, the genetically engineered host cell comprises a nucleic acid sequence encoding the heterologous silicatein fusion peptide on a bacterial F plasmid. In one embodiment, the genetically engineered host cell comprises a nucleic acid sequence encoding the heterologous silicatein fusion peptide on a bacterial expression plasmid. The expression system can further comprise an isolated nucleic acid comprising a promoter and a sequence encoding a heterologous protein operatively linked to the promoter.

In one embodiment, the isolated nucleic acid comprises a promoter that is recognized by a heterologous RNA polymerase. Accordingly, after the isolated nucleic acid is transformed into the host cell, the heterologous RNA polymerase binds to the promoter and transcribes the heterologous protein.

In one embodiment, the isolated nucleic acid is a vector. In one embodiment the vector is a plasmid. The vector may be designed to carry a gene encoding a silicatein fusion peptide operably linked to a promoter. Such a commercially available vector, having the gene encoding for a specific target protein, therefore, may be available or constructed at the customer's site, and transformed into the genetically engineered host cell of the present invention at that site.

In some embodiments, the isolated nucleic acid can be transformed into the genetically engineered host cell.

### Methods of Introduction and Expression

In one aspect, the invention provides a method for expressing a heterologous protein. In one embodiment, the method comprises providing an isolated nucleic acid comprising a promoter and a sequence encoding the heterologous protein and a genetically modified host cell of the invention. In one embodiment, the method further comprises transforming the cell with the isolated nucleic acid, incubating the cell in a media; and adding an inducer molecule to the media, wherein the addition of the inducer molecule induces expression of the silicatein fusion peptide.

The host cell can be transformed with the isolated nucleic acid by methods used to cause the uptake of DNA by living cells. The invention includes any known method of introducing DNA into living cells known or hereafter discovered. Various methods include chemical transformation (e.g., calcium chloride-mediated transformation), electroporation, sonication, macroinjection, and microinjection. See Sambrook et al., eds., Molecular Cloning: A Laboratory Manual, Cold Spring Harbor Lab Publ., 11.51, (2012) for various transformation methods.

Regardless of the method used to introduce exogenous nucleic acids into a host cell or otherwise expose a cell to the nucleic acid, in order to confirm the presence of the recombinant DNA sequence in the host cell, a variety of assays may be performed. Such assays include, for example, “molecular biological” assays well known to those of skill in the art, such as Southern and Northern blotting, reverse transcription polymerase chain reaction (RT-PCR) and PCR; “biochemical” assays, such as detecting the presence or absence of a particular peptide, e.g., by fluorescence measurements or immunological means (ELISAs and Western blots).

In some embodiments, the expression level of the heterologous protein is controlled by the amount of an inducer molecule added to the media. In some embodiments, the inducer molecule is IPTG or rhamnose. Host cells are usually cultured in conventional media as known in the art. For example, suitable growth media are common commercially prepared media such as Bacto Lactobacilli MRS broth or Agar (Difco), Luria Bertani (LB) broth, Terrific Broth (TB), Sabouraud Dextrose (SD) broth or Yeast Medium (YM) broth or E. coli chemically defined medium M9. The medium might be modified as appropriate e.g., by adding further ingredients such as buffers, salts, vitamins, cofactors or amino acids. Different media or combinations of media can be used during the culturing of the cells. In one embodiment, the medium does not include one or more molecules that could activate an inducible promoter. In some embodiments, the medium does not include glucose or lactose. Minimal medium or sugar-specific medium may allow for strict regulation of the inducible promoter region, and therefore expression of the heterologous RNA polymerase and consequently the expression of the silicatein fusion peptide.

In one embodiment, the host is Escherichia coli Top10. The culture medium is supplemented with an inducer capable of inducing the expression of heterologous polymerase from the promoter. For example, in some embodiments, the culture medium is supplemented with 0.01%-1% rhamnose to induce the expression of the heterologous polymerase from the rhaB promoter. The concentration of rhamnose in the culture medium determines the expression of heterologous polymerase, which drives the expression of said target protein and concentration of recombinant mRNA in the host cell.

Expression of the target protein is induced by the addition of inducer to the culture medium. In one embodiment, the inducer includes, but is not limited to, rhamnose, IPTG, and any combination thereof. In certain embodiments, the inducer is 0-10% rhamnose or 0-10 mM IPTG. In certain embodiments, the inducer is, 0-1% rhamnose or 0-1 mM IPTG.

In some embodiments, the expression of the silicatein fusion peptide is monitored after induction of expression. This can be monitored by e.g., SDS-PAGE combined with Coomassie/silver staining, Western-blotting or variants thereof like dot blotting (www.cshprotocols.org/). If a detectable fluorescent protein (e.g., Green Fluorescent Protein (GFP)) is fused to the silicatein fusion peptide, whole cell and in-gel fluorescence can be used to monitor expression of the target protein, fluorimeter measurements or FACs. When a membrane protein is fused to GFP, GFP is only fluorescent when the fusion is expressed in the membrane and not in inclusion bodies/aggregates (Drew et al., Protein Sci. (2005) 8, 2011-7; Drew et al., Nat. Methods (2006) 4, 303-13).

The concentration of inducer for the induction of expression of the silicatein fusion peptide can be chosen in such a way that the rate of produced recombinant mRNA and hence the rate of produced recombinant target protein is optimal for post-translational biogenesis of the silicatein fusion peptide. For example, membrane protein toxic effects of overexpression can be minimized by harmonizing translation and insertion into the membrane, and for protein requiring chaperone assistance for appropriate folding the rate of recombinant target protein production can be set to allow available chaperone(s) to keep up with the folding workload (i.e., chaperone capacity is sufficient to assist folding of the overexpressed protein).

As cell culture systems, continuous or discontinuous culture such as batch culture or fed batch culture can be applied in culture tubes, shake flasks or bacterial fermenters.

### Methods of Generating Bioglass Coated Cells

In one embodiment, the method comprises contacting a host cell comprising a nucleotide sequence encoding a silicatein fusion peptide with a molecule for mineralization by the silicatein enzyme. In some embodiments, the molecule for mineralization by the silicatein enzyme is a silicate molecule, or analog, salt or derivative. Therefore, in some embodiments, the method promotes the formation of a polysilicate layer on the surface of the cell. In some embodiments, the molecule for mineralization by the silicatein enzyme is a non-silica molecule, or analog, salt or derivative. Exemplary non-silica molecules that can be added to generate a coating with optical capture and focusing properties that can be used as a microlens include, but are not limited to titanium phosphates, titanium oxide, zirconium oxide, tin dioxide, and poly(L-lactide).

In some embodiments, the molecule for mineralization by the silicatein enzyme is a silicate molecule, or analog, salt or derivative. In some embodiments, the molecule for mineralization by the silicatein enzyme is a sodium silicate solution.

In some embodiments, the molecule for mineralization by the silicatein enzyme is orthosilicate, or analog, salt or derivative thereof. In some embodiments, the orthosilicate, or analog, salt or derivative thereof, comprises a tetraalkyl orthosilicate of formula Si(OR)₄, wherein R is an optionally substituted C1-C15 alkyl. In some embodiments, the tetraalkyl orthosilicate is tetraethyl orthosilicate (TEOS), tetramethyl orthosilicate, or tetrabutyl orthosilicate.

As used herein, the term “alkyl” includes straight-chain, branched-chain, and cyclic monovalent hydrocarbyl radicals, and combinations thereof, which contain only C and H when they are unsubstituted. The term “alkyl,” as used herein, includes cycloalkyl and cycloalkylalkyl groups. Examples include methyl, ethyl, isobutyl, cyclohexyl, cyclopentylethyl, and the like. The total number of carbon atoms in each such group is sometimes described herein, e.g., when the group can contain up to ten carbon atoms, it can be represented as 1-10C, C1-C10, C1-C10, C1-10, or C1-10. The term “heteroalkyl,” as used herein, means the corresponding hydrocarbons wherein one or more chain carbon atoms have been replaced by a heteroatom. Exemplary heteroatoms include N, O, S, and P. When heteroatoms are allowed to replace carbon atoms, for example, in heteroalkyl groups, the numbers describing the group, though still written as e.g. C3-C10, represent the sum of the number of carbon atoms in the cycle or chain plus the number of such heteroatoms that are included as replacements for carbon atoms in the cycle or chain being described.

Alkyl groups can be optionally substituted to the extent that such substitution makes sense chemically. Typical substituents include, but are not limited to, halogens (F, Cl, Br, I), ═O, ═NCN, =NOR, ═NR, OR, NR₂, SR, SO₂R, SO₂NR₂, NRSO₂R, NRCONR₂, NRC(O)OR, NRC(O)R, CN, C(O)OR, C(O)NR₂, OC(O)R, C(O)R, and NO₂, wherein each R is independently H, C₁-C₈alkyl, C₂-C₈ heteroalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, and each R is optionally substituted with halogens (F, Cl, Br, I), ═O, ═NCN, —NOR′, —NR′, OR′, NR′₂, SR′, SO₂R′, SO₂NR′₂, NR′SO₂R′, NR′CONR′₂, NR′C(O)OR′, NR′C(O)R′, CN, C(O)OR′, C(O)NR′₂, OC(O)R′, C(O)R′, and NO₂, wherein each R′ is independently H, C₁-C₈alkyl, C₂-C₈ heteroalkyl, C₁-C₈ acyl, C₂-C₈ heteroacyl, C₆-C₁₀ aryl or C₅-C₁₀ heteroaryl. Alkyl groups can also be substituted by C₁-C₈ acyl, C₂-C₈ heteroacyl, C₆-C₁₀ aryl or C₅-C₁₀ heteroaryl, each of which can be substituted by the substituents that are appropriate for the particular group.

“Optionally substituted,” as used herein, indicates that the particular group being described can have one or more hydrogen substituents replaced by a non-hydrogen substituent. In some optionally substituted groups or moieties, all hydrogen substituents are replaced by a non-hydrogen substituent (e.g., a polyfluorinated alkyl such as trifluoromethyl). If not otherwise specified, the total number of such substituents that can be present is equal to the number of H atoms present on the unsubstituted form of the group being described. Where an optional substituent is attached via a double bond, such as a carbonyl oxygen or oxo (═O), the group takes up two available valences, so the total number of substituents that may be included is reduced according to the number of available valences. As used herein, optional substituents include negatively charged groups, negatively chargeable groups, positively charged groups, positively chargeable groups, hydrophilic groups, and hydrophobic groups. In some embodiments, optional substituents include a group oxidizable to a sulfonic acid group, a thiol group (i.e., S—H), an alkylthiol group, sulfonic acid group, carboxylic acid group, amino group, and ammonium group.

In one embodiment, the method of the invention comprises providing a genetically engineered cell of the invention, incubating the cell in a media; and adding an inducer molecule for induction of expression of the silicatein fusion peptide to the media; and adding a molecule for mineralization by the silicatein enzyme to the media to promote the formation of polysilicate on the surface of the cell. In some embodiments, the molecule for mineralization by the silicatein enzyme is a silicate or non-silicate molecule that can be mineralized to form a coating having optical properties. In some embodiments, the molecule for mineralization by the silicatein enzyme is a silicate molecule, or analog, salt or derivative thereof. In some embodiments, the silicate molecule is orthosilicate, or analog, salt or derivative thereof.

### Bioglass Microlenses

In one aspect, the invention provides bioglass microlenses and methods of use thereof. In one embodiment, the bioglass microlenses of the invention comprises a layer of polysilicate generated from the formation of polysilicate on the surface of a silicatein expressing cell. In one embodiment, the bioglass microlenses of the invention comprises a layer of titanium phosphate, titanium oxide, zirconium oxide, tin dioxide, or poly(L-lactide) generated from the mineralization of precursor molecules on the surface of a silicatein expressing cell.

In some embodiments, the bioglass microlenses of the invention comprises a spherical, cylindrical, or ovoid structure of polysilicate having a thickness of 10-30 nm. In some embodiments, the size of the spherical, cylindrical, or ovoid structure of polysilicate is dependent on the size of the silicatein expressing cell(s) used to generate the microlenses. For example, in some embodiments, when the silicatein expressing cell is an E. coli cell, the microlens is a 3-Dimensional structure of polysilicate approximately 0.5-3.0 micrometers long, with a radius of about 0.25-1.0 micrometers, and a thickness of about 10-30 nm. In some embodiments, when the silicatein expressing cell is an E. coli cell overexpressing BolA, the microlens is a 3-Dimensional structure of polysilicate approximately 0.5-1.5 micrometers long, with radius or width of about 0.5-1.0 micrometers, and a thickness of about 10-30 nm. In some embodiments, when the silicatein expressing cell is an E. coli cell overexpressing SulA, the microlens is a 3-Dimensional structure of polysilicate approximately 1.0-100 micrometers long, with radius or width of about 0.5-1.0 micrometers, and a thickness of about 10-30 nm. However, the invention is not limited to a microlens comprising the above dimensions as a person of skill in the art would understand that the dimensions of the microlenses of the invention are dependent on the dimensions of the host cell(s), as well as the gene being expressed. For example, in some embodiments, a host cell overexpressing CrvAB from Vibrio cholerae has increased curvature.

In some embodiments, the invention provides for use of the bioglass microlenses of the invention. Uses include, but are not limited to incorporation into optical devices, solar cells, compact image sensors, 3D displays, concentrators for photovoltaics, flexible image sensors, flexible substrates with optical activity, one-photon and multiphoton microscopy, super-resolution emission depletion microscopy (STED), and optogenetic stimulation and readouts.

In some embodiments, the invention provides methods of generating devices comprising at least one bioglass microlens. In some embodiments, the method comprises coating a surface or substrate, or a portion thereof, with silicatein expressing cells of the invention, and contacting the coated surface, substrate, or portion thereof, with a silicat molecule, or analog, salt or derivative thereof to induce the formation of a polysilicate layer on the surface of the silicatein expressing cells, thereby generating at least one bioglass microlense. In some embodiments, the surface, substrate, or portion thereof is coated with at least 100, 1000, 10,000, 100,000, 1×10\hat{ }6, 1×10\hat{ }7, 1×10\hat{ }8, 1×10\hat{ }9 or more than 1×10\hat{ }9 individual silicatein expressing cells, for the generation of an array of bioglass microlenses on the surface or substrate. In some embodiments, the individual silicatein expressing cells are size selected prior to coating the surface or substrate to ensure the generation of a uniform array of bioglass microlenses on the surface or substrate.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Without further description, it is believed that one of ordinary skill in the art can, using the preceding description and the following illustrative examples, make and utilize the present invention and practice the claimed methods. The following working examples therefore, point out specific embodiments of the present invention, and are not to be construed as limiting in any way the remainder of the disclosure.

### Example 1: Biological Methods of Manufacturing Bioglasses with Unique Physical and Chemical Properties

Silicatein can catalyze the templating and polymerization of silica monomers (monosilicate) at physiological pH and temperature, resulting in the creation of amorphous silica nanospheres (polysilicate) from soluble precursors (Schroder et al., 2008, Nat Prod Rep, 25 (3), 455-74). The resultant bioglass structures found in nature show ultra-precise control over the morphology of the structure, forming elaborately shaped spicules that harbor a proteinaceous axial filament in a central canal surrounded by concentric layers of polysilicate with fiber optic properties (Sundar et al., 2003, Nature, 424 (6951), 899-900; Schroder et al., 2008, Nat Prod Rep, 25 (3), 455-74). The data presented herein demonstrate that expression of silicatein can be harnessed to fabricate polysilicate particles with a range of powerful optical properties, with control at the micro- and nano-scale.

### Cloning of Silicatein Constructs and Transformation of Bacteria

Bacterial expression constructs coding various outer membrane protein fusion peptides of silicatein were generated by PCT method as described (Curnow, P., et al., 2006, Journal of the American Chemical Society, 45 (4): 613-616).

The PCR products of RBS, OmpA, and Sdom were inserted into the bacterial expression vector, pRHA113, to produce OmpASdom fusion proteins. The plasmids were then delivered into chemically competent Top10 E. coli.

### Induction of Silicatein Fusion Proteins and Microlens Biosynthesis

A culture of OmpASdom-expressing E. coli was incubated at 37° C. with 0.2% rhamnose (w/v) for 3 hours. Orthosilicate was added to the culture to a final concentration of 60 μM and the culture incubated for an additional 3 hours, after which cells were harvested.

### Rhodamine Staining of Bio-Glass Microlenses

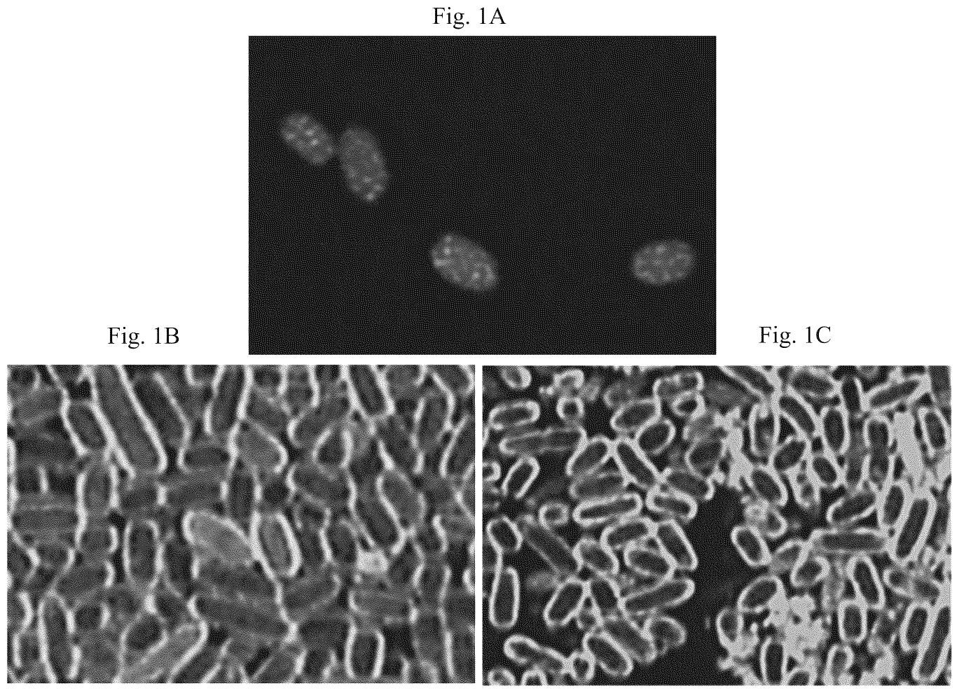

Cells with bio-glass microlenses were suspended in culture medium and 1/1000 (v/v) Rhodamine123 was added. The culture was incubated for 10 minutes, after which cells were harvested, rinsed, and examined under fluorescent microscopy at an excitation wavelength of 545 nm as can be seen in FIG. 1.

### Examination of Cell Shape

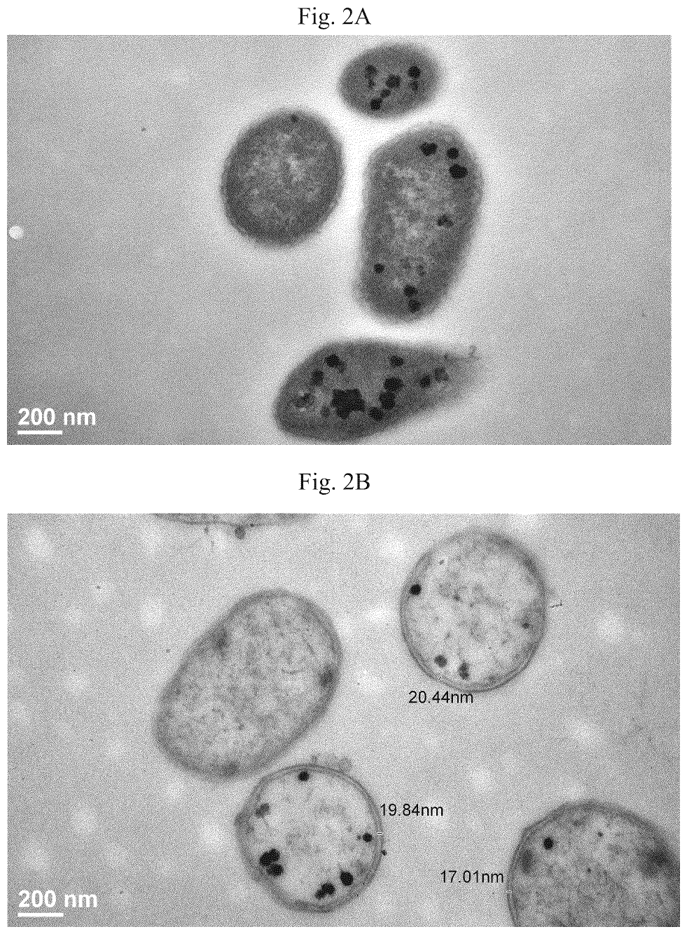

Cell shape was examined through transmission electron microscopy (TEM) and thickness of the bio-glass microlenses calculated as can be seen in FIG. 2.

### Quantification of Lensing Behaviors

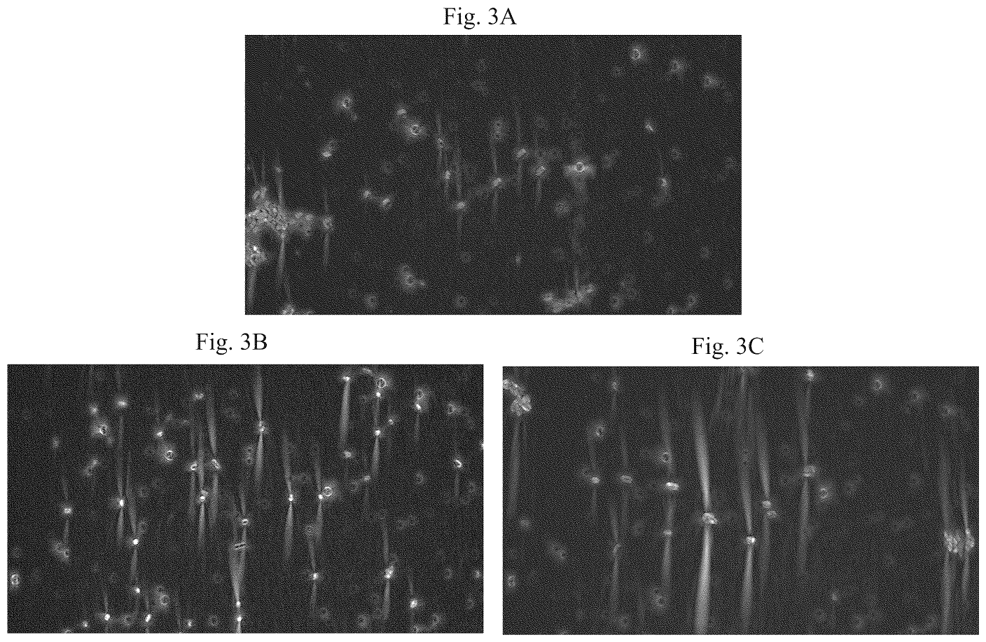

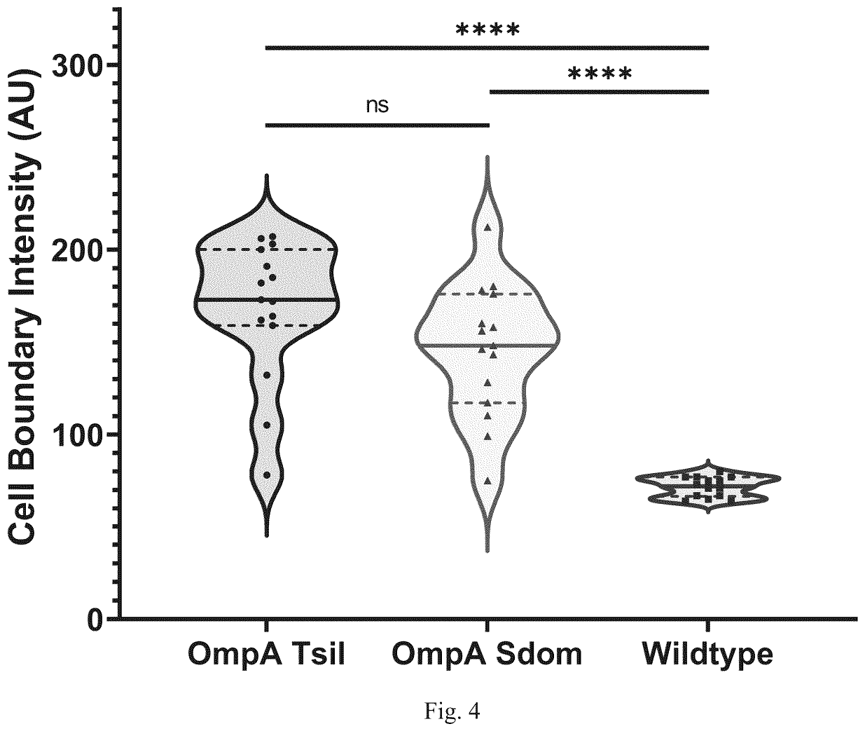

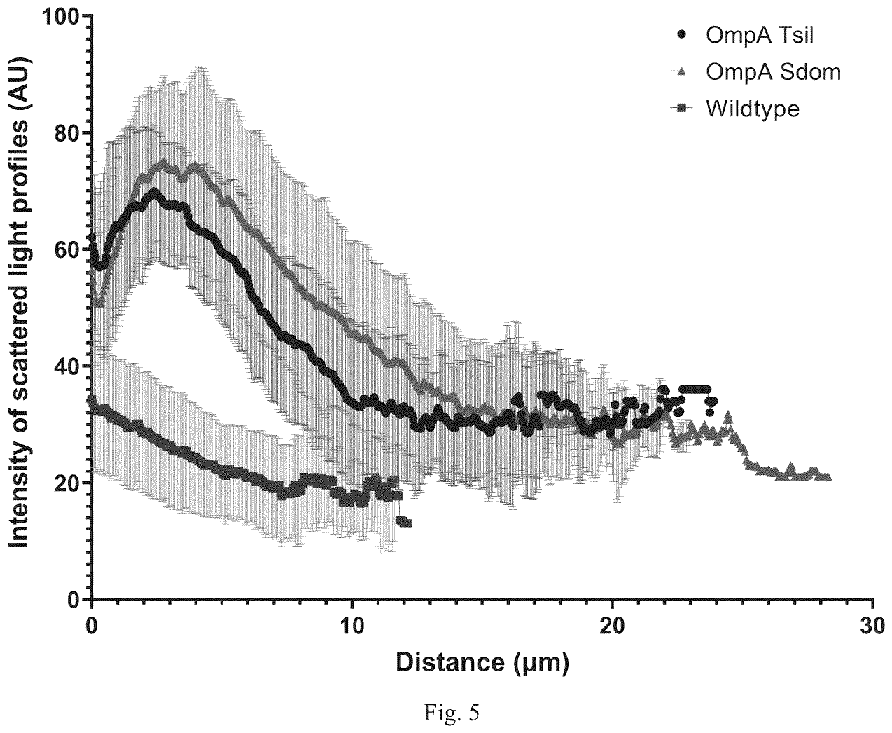

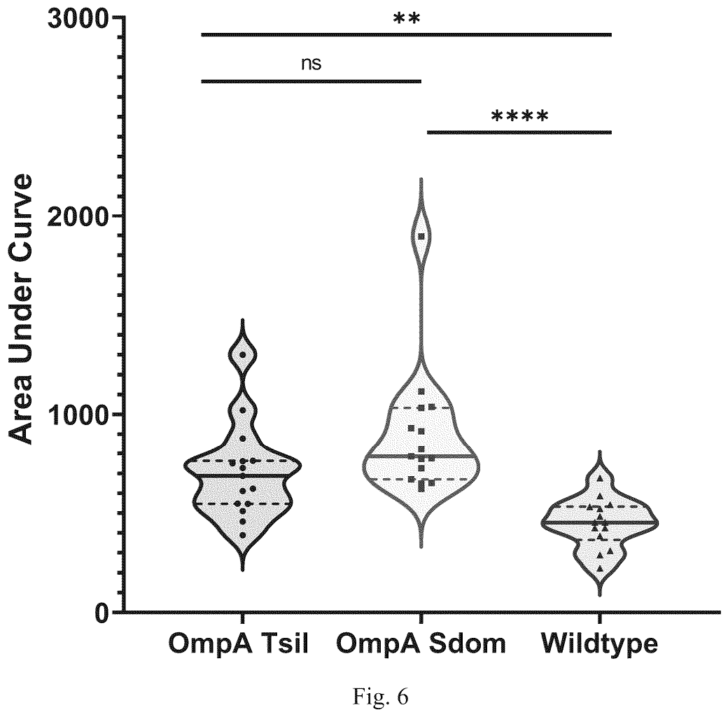

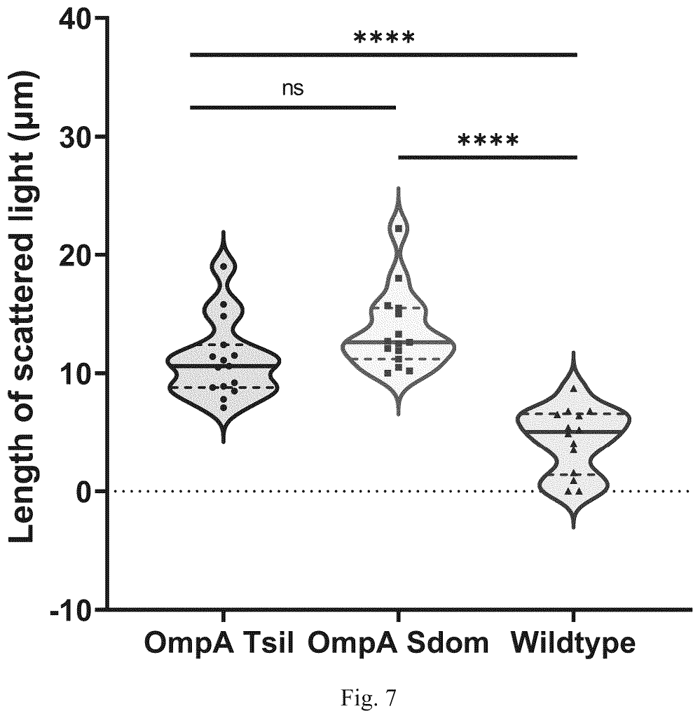

Alexa488 amine dye was added to 0.1% poly-L-lysine 1% agarose pads at a concentration of 50 μL/pad. The pads were rinsed of excess dye with 1M glycine and cells added to the pad. The lensing behaviors of the bio-glass-encapsulated cells were examined for cell body intensity, plot profile, total light scattered (AUC), and length of scattering through laser-illumination microscopy as depicted in FIG. 3 through FIG. 7.

### Chemical Induction of Spherical Bio-Glass Microlens Shape

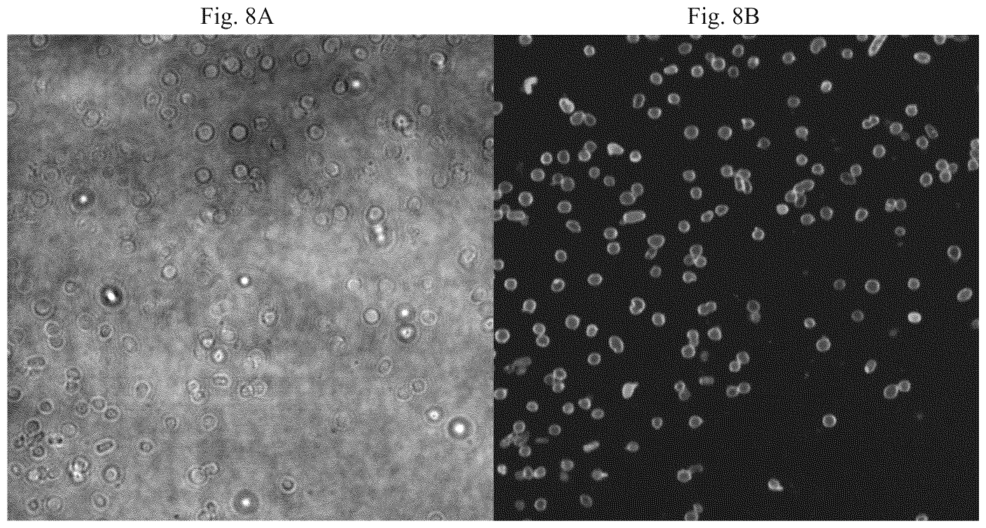

E. coli that had been transformed with the OmpASdom plasmid were incubated at 37° C. with 0.2% rhamnose (w/v) and S-(3,4-dichlorobenzyl) isothiourea (A22) at a concentration of 10 μg/mL for three hours, after which orthosilicate was added to a final concentration of 60 μM. The culture was incubated an additional three hours, after which cells were harvested, rinsed, and stained with Rhodamine123 at 1/1000 (v/v), and examined under brightfield and fluorescent microscopy as depicted in FIG. 8.

### Genetic Induction of Spherical Bio-Glass Microlens Shape

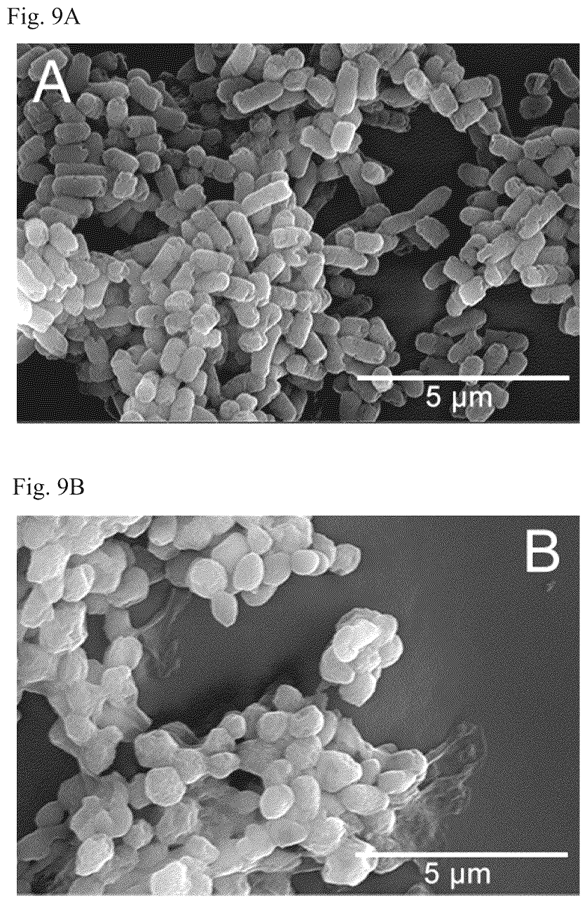

Top10 E. coli that had been transformed with both the OmpASdom plasmid and BolA plasmid were incubated at 37° C. with 0.2% rhamnose (w/v) for three hours, after which orthosilicate was added to a final concentration of 60 μM and incubated for three more hours, after which the cells were harvested and examined under scanning electron microscopy (SEM) as depicted in FIG. 9 in which, panel A shows wild-type cells expressing OmpASdom, and panel B shows wild-type cells expressing both OmpASdom and BolA. These cells were incubated with orthosilicate, so the images in FIG. 9 are of bioglass-coated cells.

### Elongation of Bio-Glass Microlenses

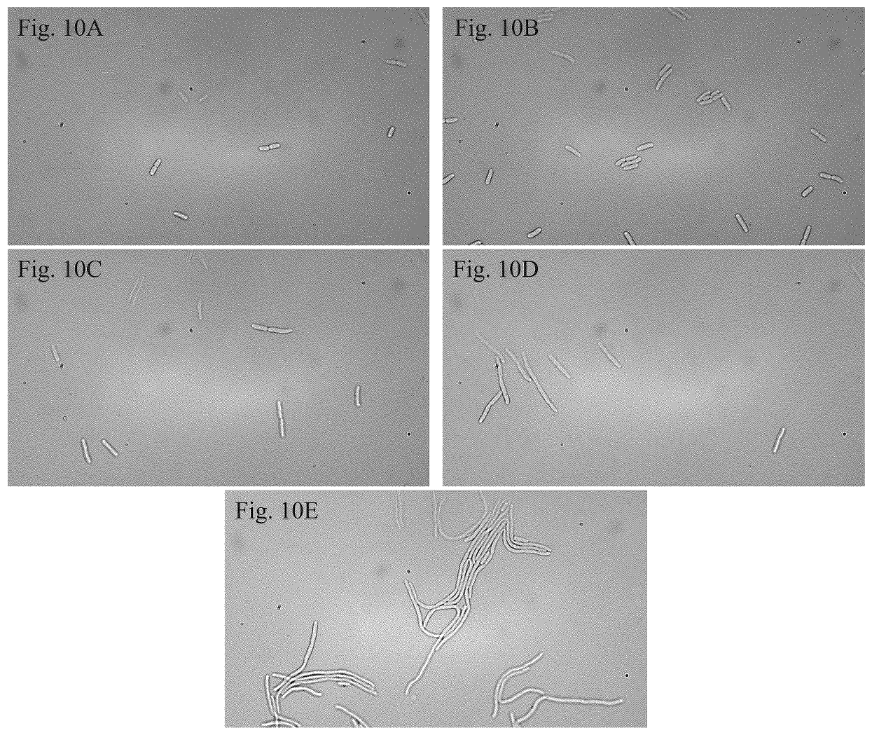

Top10 E. coli were incubated with cephalexin at a concentration of 25 μg/mL for up to three hours. Elongation of cell shape can be seen in FIG. 10.

Genetic Induction of Elongation of E. coli

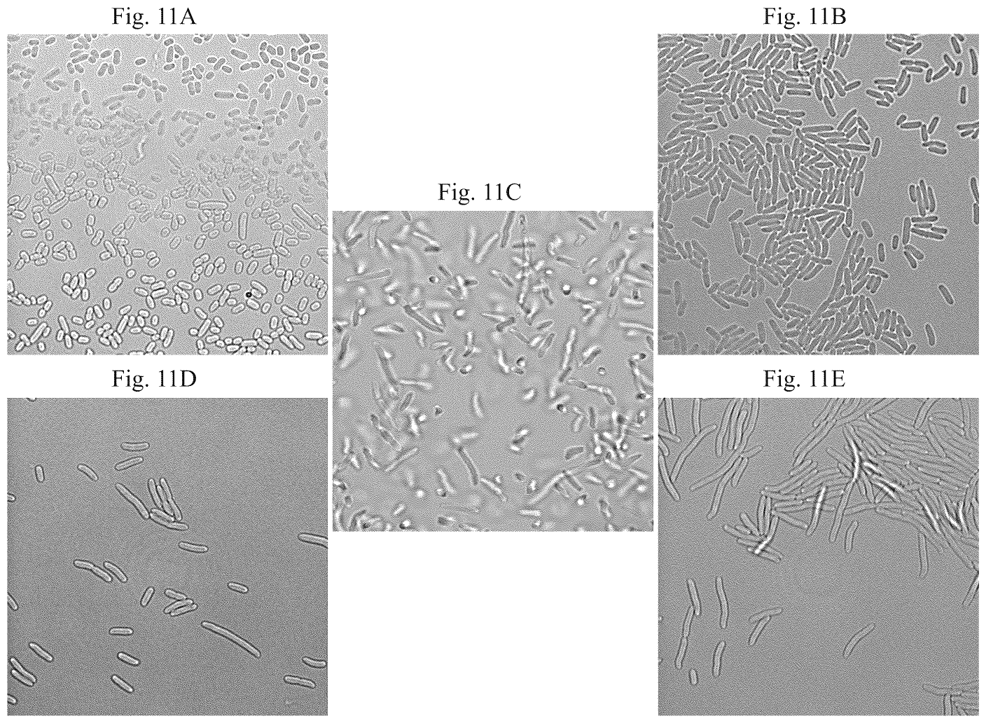

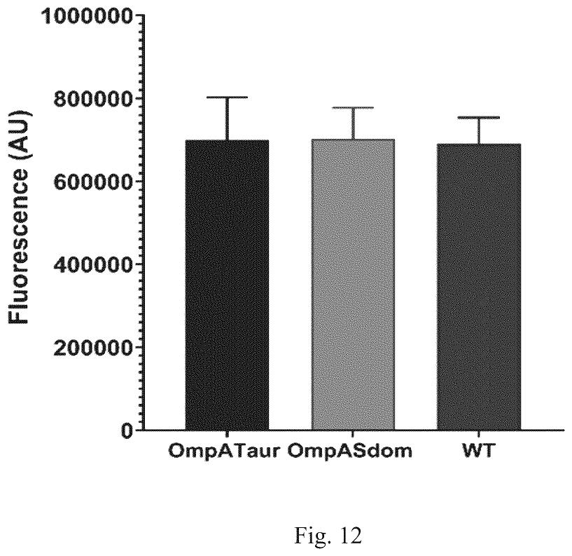

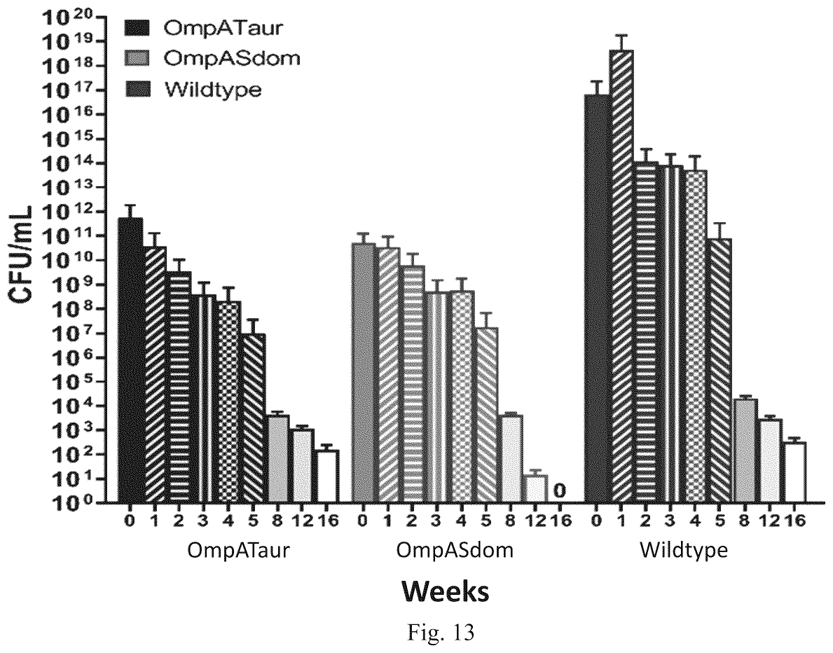

Top10 E. coli that had been transformed with a SulA plasmid were incubated at 37° C. with for up to five hours. Elongation of cell shape can be seen in FIG. 11.

### Microlens Array

To fabricate biological-based microlens arrays for applications such as light harvesting for solar cells, the bacteria cells are ordered and attached to a surface. Silicatein-expressing E. coli adhere to lattice surfaces coated in a patterned layer of silica- or silicatein-binding peptides using clean-room lithography techniques. The specific adherence and arrangement of the bioglass-coated bacteria is assayed through co-expression of a fluorescent protein (such as GFP) in the bacteria and detection using confocal microscopy. The lattice arrangement provides exquisite control over the spatial pattern and therefore the optical properties of the selectively deposited material. The optical properties of the microlens arrays, the light-focusing properties, the effects of bacterial silicatein expression levels, the microlens shapes, and the spatial pattern of microlens deposition are then analyzed.

The fabrication of microlenses using the bacterial platform finds application in optical and biomedical industries. The advanced optical materials that are developed are suitable for a wide range of applications, including compact image sensors, 3D displays, concentrators for photovoltaics, and solar cell technologies. This approach reduces energy consumption and waste as compared to the currently used lithography-based methods for microlens production, a problem that is currently holding back widespread adoption of printed solar cells (Cheng et al., 2016, Nature, 539 (7630), 488-489). Additionally, the bacterial microlenses are 10-100× smaller than conventional microlenses, which are currently difficult and costly to produce below a size range of tens or hundreds of micrometers (Krupenkin et al., 2003, Appl Phys Lett, 82 (3), 316-318). This breakthrough in microlens miniaturization allows for more lenses to be included in each array, which results in a dramatic increase in the amount of light that can be captured. The smaller size of the microlenses also provides new opportunities to be deposited onto flexible substrates while maintaining the integrity of the optical activity. The average size of photodetectors in a photovoltaic cell is in the nano- to micrometer range (Yang et al., 2005, Nat Mater, 4 (1), 37-41). Since bacterial microlenses approach this size for the first time, a much greater amount of the light that they focus can be captured compared to conventional microlenses. This advance could be applied to producing a new generation of solar panels that can generate substantially more electricity, while being produced in a more environmentally-friendly manner.

### Example 2: Bioglass Encapsulation of Bacteria Cells does not Affect their Metabolic Activity

An alamarBlue assay was performed on wild-type E. coli cells, as well as E. coli cells expressing OmpA-silicatein constructs, where the silicatein enzyme was derived either from T. aurantia or S. domuncula. The fluorescence level of the cells is an indicator of metabolic activity. The assay showed no significant differences in metabolic activity between the different cell types, indicating that the bioglass-encapsulated cells had similar metabolic activity to unencapsulated wild-type cells.

### Methods:

Wild-type bacterial cells and bioglass-encapsulated bacterial cells were diluted to an OD600 of 0.1 in fresh LB (plus Ampicillin [100 μg/mL] for bioglass-encapsulated cells) or diluted further to an OD600 of 0.01 in 1× TBS. 100 μL of each sample was added to wells of a 96-well plate and then 10 μL alamarBlue reagent was added to the cells. The plate was immediately placed in the plate reader where fluorescence readings occurred every 15 minutes for 24 hours. AlamarBlue excitation: 530 nm; emission: 590 nm. As the cells reduce alamarBlue, it changes from blue to highly red fluorescent. OD600 readings were taken at the same intervals as well on replicate samples not containing alamarBlue. Fluorescence data was calculated using the 1× TBS diluted samples, and the OD600 data was calculated using the LB diluted samples.

### Example 3: Bioglass Encapsulation of Bacteria Cells Results in 10,000× Fewer Dividing Cells

A colony-forming unit (CFU) experiment was performed on wild-type E. coli cells, as well as E. coli cells expressing OmpA-silicatein constructs, where the silicatein enzyme was derived either from T. aurantia or S. domuncula. The number of CFUs measured for each sample is an indication of how many bacteria cells are able to divide. Wild-type cells showed CFU levels 10,000 times higher than those of the two bioglass-encapsulated strains, indicating that bioglass-encapsulated cells are dramatically hindered in their ability to divide.

### Methods:

Wild-type bacterial cells and bioglass-encapsulated bacterial cells at an OD600 of 0.1 were serially diluted in 1× TBS. These samples were plated in triplicate on LB (plus Ampicillin [100 μg/mL] for bioglass-encapsulated cells) agar plates-100 μL of sample per plate was used. The plates were grown overnight at 37° C., and the number of colonies were counted the following morning. To obtain CFU/mL the following equation was used: #colonies dilution/mL of sample plated.

### Example 4: SEQUENCES

Suberites domuncula Silicatein (BBa_K1890050)
Nucleotide
(SEQ ID NO: 1)
Atggattaccccgaggcagtggattggcgcactaagggggcagttactgcggtgaaggatcagggaga
ctgtggtgcatcatacgcatttagcgccatgggtgccttggaaggcgcgaacgccttagcgaaaggcaacgccgtatccctgtc
ggagcaaaacatcattgattgttcaattccttatggtaaccatggttgtcatggtggaaacatgtatgatgctttcttatacgtcatcgc
gaatgagggagtggaccaagattcagcgtatcctttcgtcgggaaacagtcctcttgtaactacaactcgaagtataagggcacg
tcgatgagtggtatggtgtctattaagtcgggatcagagtccgatttgcaagcagccgtctctaatgtcggcccagtttcggtggca
attgacggcgcaaactcggccttccgcttttattattctggcgtctatgattccagccgttgttctagttcgtctttgaatcatgcaatg
gtggtgacggggtacggcagttacaatggaaaaaaatactggcttgccaagaactcctggggcactaactgggggaactcagg
gtacgttatgatggctcgtaataaatacaatcagtgcggtattgcgacggacgcatcctatcctacgttgtaa
Amino Acid
(SEQ ID NO: 2)
MDYPEAVDWRTKGAVTAVKDQGDCGASYAFSAMGALEGANAL
AKGNAVSLSEQNIIDCSIPYGNHGCHGGNMYDAFLYVIANEGVDQDSAYPFVGK
QSSCNYNSKYKGTSMSGMVSIKSGSESDLQAAVSNVGPVSVAIDGANSAFRFYY
SGVYDSSRCSSSSLNHAMVVTGYGSYNGKKYWLAKNSWGTNWGNSGYVMMA
RNKYNQCGIATDASYPTL
Tethya aurantia Silicatein (BBa_K1890051)
Nucleotide
(SEQ ID NO: 3)
Atgtatctcggcacgttggttgttttgtgtgttttgggggctgctattggagagccaatgcctcagtatgagtt
caaggaggaatggcagctgtggaagaaacaacatgacaagtcttacagcaccaacttggaggaactggagaaacatcttgtct
ggctctccaacaagaagtacattgaactgcacaatgccaatgcagacacctttggattcactctagctatgaaccatctaggagat
atgactgaccatgaatacaaggagagatacctcacatacactaacagcaaatctggtaactacaccaaggtgttcaaacgtgagc
catggatggcctacccggagactgtagattggagaacaaagggcgctgtgactggtatcaagagccagggagattgtggtgcc
agctatgcattcagtgccatgggtgcacttgaaggaatcaatgcacttgctactggaaagctgacctatctcagtgaacagaacat
cattgattgctctgtaccttatggtaaccatggttgcaagggtggaaacatgtatgtggctttcctctatgttgttgctaacgaaggag
ttgatgatgggggttcctatccatttagaggaaagcaatccagttgtacgtatcaagagcagtaccgtggtgcaagtatgtctggct
cagttcaaatcaacagtggtagtgaatctgatctggaagcagctgtagccaatgttggtccagttgcagtagctattgatggagag
tcaaatgctttcagattctattacagtggagtgtacgactcctccagatgttctagtagcagtctcaaccacgccatggtgatcactg
gctatggaatttcaaataaccaggaatactggcttgcaaagaacagctggggtgagaactggggagaactgggctatgtgaaga
tggccaggaacaagtacaatcaatgtgggattgctagtgatgcctcctaccccactctctag
Amino Acid
(SEQ ID NO: 4)
MYLGTLVVLCVLGAAIGEPMPQYEFKEEWQLWKKQHDKSYSTNL
EELEKHLVWLSNKKYIELHNANADTFGFTLAMNHLGDMTDHEYKERYLTYTNS
KSGNYTKVFKREPWMAYPETVDWRTKGAVTGIKSQGDCGASYAFSAMGALEGI
NALATGKLTYLSEQNIIDCSVPYGNHGCKGGNMYVAFLYVVANEGVDDGGSYP
FRGKQSSCTYQEQYRGASMSGSVQINSGSESDLEAAVANVGPVAVAIDGESNAF
RFYYSGVYDSSRCSSSSLNHAMVITGYGISNNQEYWLAKNSWGENWGELGYVK
MARNKYNQCGIASDASYPTL
OmpA (BBa_K1890052)
Nucleotide
(SEQ ID NO: 5)
Atgaaggctacaaaattggtgttgggagcggtaatcctgggttcgactctgttggcaggctgttcttccaat
gcgaagatcgaccagttatccagtgatgtggggatcaacccatacgtcggtttcgagatgggttatgactggctgggccgcatgc
catataagggatccgtggaaaacggcgcatataaggcacaaggagtccaattgaccgcaaagttgggttaccccattactgatg
atttggacatctacacgcgtctgggtgggatggtgtggcgcgcagacacaaaaagtaatgtttatggtaagaatcacgatacagg
agtttctcccgtttttgctggaggagtggagtatgcgatcacgccggaaatcgcgacgcgcttggagtaccaatggactaacaac
attggcgacgcgcacacgattggcactcgtcctgacaacggcatcccaggt
Amino Acid
(SEQ ID NO: 6)
MKATKLVLGAVILGSTLLAGCSSNAKIDQLSSDVGINPYVGFEMG
YDWLGRMPYKGSVENGAYKAQGVOLTAKLGYPITDDLDIYTRLGGMVWRADT
KSNVYGKNHDTGVSPVFAGGVEYAITPEIATRLEYQWTNNIGDAHTIGTRPDNGI
PG
INP (BBa_K1890053)
Nucleotide
(SEQ ID NO: 7)
Atgaccttagataaagccttggtattacgcacgtgtgctaacaatatggcagatcactgcgggttaatttgg
ccggccagcggtactgtggagagccgctattggcagagtacccgccgccacgaaaacgggcttgttggccttctgtggggag
ccggtacgtctgcctttctgtccgtccatgccgatgcgcgttggatcgtctgcgaagtcgccgtagcggacatcatttcgttggag
gaaccgggcatggtcaaattcccccgcgctgaagttgttcacgtgggtgatcgcatttcagcttcacacttcatctcggcacgcca
agccgatcccgcaagcacgagcaccagcacttcaacttccaccttaacgcctatgccgaccgcgattccgacaccgatgccgg
ccgttgcatcggttactctgccggtcgccgagcaggcccgccatgaagttttcgacgtagcgagtgtttcggccgccgctgccc
ccgtgaatactctgcctgtcactacgcctcagaaccttcagactgcgacttacgggagcacattatctggggataaccactctcgc
ttaatcttccgtttgtgggacggtaaacgttatcgccaacttgtggctcgtaccggcgagaacggggttgaggctgacatcccgta
ctatgtgaacgaggacgacgatattgttgataagccggacgaggacgacgactggatcgaggtgaag
Amino Acid
(SEQ ID NO: 8)
MTLDKALVLRTCANNMADHCGLIWPASGTVESRYWQSTRRHEN
GLVGLLWGAGTSAFLSVHADARWIVCEVAVADIISLEEPGMVKFPRAEVVHVGD
RISASHFISARQADPASTSTSTSTSTLTPMPTAIPTPMPAVASVTLPVAEQARHEVF
DVASVSAAAAPVNTLPVTTPQNLQTATYGSTLSGDNHSRLIFRLWDGKRYRQLV
ARTGENGVEADIPYYVNEDDDIVDKPDEDDDWIEVK
Promoter (lac UV5 promoter)
(SEQ ID NO: 9)
tttacactttatgcttccggctcgtataatg
Promoter (rhaB promoter from pRHA113)
(SEQ ID NO: 10)
CACCACAATTCAGCAAATTGTGAACATCATCACGTTCATCTTTC
CCTGGTTGCCAATGGCCCATTTTCCTGTCAGTAACGAGAAGGTCGCGAATTCA
GGCGCTTTTTAGACTGGTCGTA
RBS (BBa_B0034)
(SEQ ID NO: 11)
aaagaggagaaa
Double Terminator (rrn T1 terminator and T7Te terminator)
(SEQ ID NO: 12)
Caaataaaacgaaaggctcagtcgaaagactgggcctttcgttttatctgttgtttgtcggtgaacgctctct
actagagtcacactggctcaccttcgggtgggcctttctgcg
OmpA Suberites domuncula Silicatein in pRHA113
Promoter-RBS-OmpASdomSilicatein
coding sequence for OmpA is bold, coding sequence for SdomSilicatein is underlined
(SEQ ID NO: 13)
CACCACAATTCAGCAAATTGTGAACATCATCACGTTCATCTTTC
CCTGGTTGCCAATGGCCCATTTTCCTGTCAGTAACGAGAAGGTCGCGAATTCA
GGCGCTTTTTAGACTGGTCGTAATGAAATTCAGGAGGTTGTCGACTCTAGAtac
tagagaaagaggagaaatactagatgaaggctacaaaattggtgttgggagcggtaatcctgggttcgactctgttggcag
gctgttcttccaatgcgaagatcgaccagttatccagtgatgtggggatcaacccatacgtcggtttcgagatgggttatg
actggctgggccgcatgccatataagggatccgtggaaaacggcgcatataaggcacaaggagtccaattgaccgcaa
agttgggttaccccattactgatgatttggacatctacacgcgtctgggtgggatggtgtggcgcgcagacacaaaaagt
aatgtttatggtaagaatcacgatacaggagtttctcccgtttttgctggaggagtggagtatgcgatcacgccggaaatc
gcgacgcgcttggagtaccaatggactaacaacattggcgacgcgcacacgattggcactcgtcctgacaacggcatcc
caggtatggattaccccgaggcagtggattggcgcactaagggggcagttactgcggtgaaggatcagggagactgtggtgc
atcatacgcatttagcgccatgggtgccttggaaggcgcgaacgccttagcgaaaggcaacgccgtatccctgtcggagcaaa
acatcattgattgttcaattccttatggtaaccatggttgtcatggtggaaacatgtatgatgctttcttatacgtcatcgcgaatgagg
gagtggaccaagattcagcgtatcctttcgtcgggaaacagtcctcttgtaactacaactcgaagtataagggcacgtcgatgagt
ggtatggtgtctattaagtcgggatcagagtccgatttgcaagcagccgtctctaatgtcggcccagtttcggtggcaattgacgg
cgcaaactcggccttccgcttttattattctggcgtctatgattccagccgttgttctagttcgtctttgaatcatgcaatggtggtgac
ggggtacggcagttacaatggaaaaaaatactggcttgccaagaactcctggggcactaactgggggaactcagggtacgttat
gatggctcgtaataaatacaatcagtgcggtattgcgacggacgcatcctatcctacgttgtaa
OmpA-Sdom Silicatein
OmpA sequence is bold, SdomSilicatein is underlined
(SEQ ID NO: 14)
MKATKLVLGAVILGSTLLAGCSSNAKIDQLSSDVGINPYVGFE
MGYDWLGRMPYKGSVENGAYKAQGVOLTAKLGYPITDDLDIYTRLGGMV
WRADTKSNVYGKNHDTGVSPVFAGGVEYAITPEIATRLEYQWTNNIGDAHT
IGTRPDNGIPGMDYPEAVDWRTKGAVTAVKDQGDCGASYAFSAMGALEGAN
ALAKGNAVSLSEQNIIDCSIPYGNHGCHGGNMYDAFLYVIANEGVDQDSAYPFV
GKQSSCNYNSKYKGTSMSGMVSIKSGSESDLQAAVSNVGPVSVAIDGANSAFRF
YYSGVYDSSRCSSSSLNHAMVVTGYGSYNGKKYWLAKNSWGTNWGNSGYVM
MARNKYNQCGIATDASYPTL
Full Vector Map (pRHA113 vector backbone containing-rhaR, rhaS, rhaB promoter,
RBS, OmpA S. dom Silicatein, rop, pUC origin, AmpR, AmpR promoter)
coding sequence for OmpA is bold, coding sequence for SdomSilicatein is
underlined
(SEQ ID NO: 15)
TTCTCATGTTTGACAGCTTATCATCGATAAGCTTAATTAATCTTT
CTGCGAATTGAGATGACGCCACTGGCTGGGCGTCATCCCGGTTTCCCGGGTA
AACACCACCGAAAAATAGTTACTATCTTCAAAGCCACATTCGGTCGAAATAT
CACTGATTAACAGGCGGCTATGCTGGAGAAGATATTGCGCATGACACACTCT
GACCTGTCGCAGATATTGATTGATGGTCATTCCAGTCTGCTGGCGAAATTGCT
GACGCAAAACGCGCTCACTGCACGATGCCTCATCACAAAATTTATCCAGCGC
AAAGGGACTTTTCAGGCTAGCCGCCAGCCGGGTAATCAGCTTATCCAGCAAC
GTTTCGCTGGATGTTGGCGGCAACGAATCACTGGTGTAACGATGGCGATTCA
GCAACATCACCAACTGCCCGAACAGCAACTCAGCCATTTCGTTAGCAAACGG
CACATGCTGACTACTTTCATGCTCAAGCTGACCGATAACCTGCCGCGCCTGCG
CCATCCCCATGCTACCTAAGCGCCAGTGTGGTTGCCCTGCGCTGGCGTTAAAT
CCCGGAATCGCCCCCTGCCAGTCAAGATTCAGCTTCAGACGCTCCGGGCAAT
AAATAATATTCTGCAAAACCAGATCGTTAACGGAAGCGTAGGAGTGTTTATC
GTCAGCATGAATGTAAAAGAGATCGCCACGGGTAATGCGATAAGGGCGATC
GTTGAGTACATGCAGGCCATTACCGCGCCAGACAATCACCAGCTCACAAAAA
TCATGTGTATGTTCAGCAAAGACATCTTGCGGATAACGGTCAGCCACAGCGA
CTGCCTGCTGGTCGCTGGCAAAAAAATCATCTTTGAGAAGTTTTAACTGATGC
GCCACCGTGGCTACCTCGGCCAGAGAACGAAGTTGATTATTCGCAATATGGC
GTACAAATACGTTGAGAAGATTCGCGTTATTGCAGAAAGCCATCCCGTCCCT
GGCGAATATCACGCGGTGACCAGTTAAACTCTCGGCGAAAAAGCGTCGAAAA
GTGGTTACTGTCGCTGAATCCACAGCGATAGGCGATGTCAGTAACGCTGGCC
TCGCTGTGGCGTAGCAGATGTCGGGCTTTCATCAGTCGCAGGCGGTTCAGGT
ATCGCTGAGGCGTCAGTCCCGTTTGCTGCTTAAGCTGCCGATGTAGCGTACGC
AGTGAAAGAGAAAATTGATCCGCCACGGCATCCCAATTCACCTCATCGGCAA
AATGGTCCTCCAGCCAGGCCAGAAGCAAGTTGAGACGTGATGCGCTGTTTTC
CAGGTTCTCCTGCAAACTGCTTTTACGCAGCAAGAGCAGTAATTGCATAAAC
AAGATCTCGCGACTGGCGGTCGAGGGTAAATCATTTTCCCCTTCCTGCTGTTC
CATCTGTGCAACCAGCTGTCGCACCTGCTGCAATACGCTGTGGTTAACGCGCC
AGTGAGACGGATACTGCCCATCCAGCTCTTGTGGCAGCAACTGATTCAGCCC
GGCGAGAAACTGAAATCGATCCGGCGAGCGATACAGCACATTGGTCAGACA
CAGATTATCGGTATGTTCATACAGATGCCGATCATGATCGCGTACGAAACAG
ACCGTGCCACCGGTGATGGTATAGGGCTGCCCATTAAACACATGAATACCCG
TGCCATGTTCGACAATCACAATTTCATGAAAATCATGATGATGTTCAGGAAA
ATCCGCCTGCGGGAGCCGGGGTTCTATCGCCACGGACGCGTTACCAGACGGA
AAAAAATCCACACTATGTAATACGGTCATACTGGCCTCCTGATGTCGTCAAC
ACGGCGAAATAGTAATCACGAGGTCAGGTTCTTACCTTAAATTTTCGACGGA
AAACCACGTAAAAAACGTCGATTTTTCAAGATACAGCGTGAATTTTCAGGAA
ATGCGGTGAGCATCACATCACCACAATTCAGCAAATTGTGAACATCATCACG
TTCATCTTTCCCTGGTTGCCAATGGCCCATTTTCCTGTCAGTAACGAGAAGGT
CGCGAATTCAGGCGCTTTTTAGACTGGTCGTAATGAAATTCAGGAGGTTGTCG
ACTCTAGAtactagagaaagaggagaaatactagatgaaggctacaaaattggtgttgggagcggtaatcctgggtt
cgactctgttggcaggctgttcttccaatgcgaagatcgaccagttatccagtgatgtggggatcaacccatacgtcggtt
tcgagatgggttatgactggctgggccgcatgccatataagggatccgtggaaaacggcgcatataaggcacaaggag
tccaattgaccgcaaagttgggttaccccattactgatgatttggacatctacacgcgtctgggtgggatggtgtggcgcg
cagacacaaaaagtaatgtttatggtaagaatcacgatacaggagtttctcccgtttttgctggaggagtggagtatgcg
atcacgccggaaatcgcgacgcgcttggagtaccaatggactaacaacattggcgacgcgcacacgattggcactcgtc
ctgacaacggcatcccaggtatggattaccccgaggcagtggattggcgcactaagggggcagttactgcggtgaaggatca
gggagactgtggtgcatcatacgcatttagcgccatgggtgccttggaaggcgcgaacgccttagcgaaaggcaacgccgtat
ccctgtcggagcaaaacatcattgattgttcaattccttatggtaaccatggttgtcatggtggaaacatgtatgatgctttcttatacg
tcatcgcgaatgagggagtggaccaagattcagcgtatcctttcgtcgggaaacagtcctcttgtaactacaactcgaagtataag
ggcacgtcgatgagtggtatggtgtctattaagtcgggatcagagtccgatttgcaagcagccgtctctaatgtcggcccagtttc
ggtggcaattgacggcgcaaactcggccttccgcttttattattctggcgtctatgattccagccgttgttctagttcgtctttgaatca
tgcaatggtggtgacggggtacggcagttacaatggaaaaaaatactggcttgccaagaactcctggggcactaactggggga
actcagggtacgttatgatggctcgtaataaatacaatcagtgcggtattgcgacggacgcatcctatcctacgttgtaaGGTA
CCTCGACCGATGCCCTTGAGAGCCTTCAACCCAGTCAGCTCCTTCCGGTGGGC
GCGGGGCATGACTATCGTCGCCGCACTTATGACTGTCTTCTTTATCATGCAAC
TCGTAGGACAGGTGCCGGCAGCGCTCTGGGTCATTTTCGGCGAGGACCGCTT
TCGCTGGAGCGCGACGATGATCGGCCTGTCGCTTGCGGTATTCGGAATCTTGC
ACGCCCTCGCTCAAGCCTTCGTCACTGGTCCCGCCACCAAACGTTTCGGCGAG
AAGCAGGCCATTATCGCCGGCATGGCGGCCGACGCGCTGGGCTACGTCTTGC
TGGCGTTCGCGACGCGAGGCTGGATGGCCTTCCCCATTATGATTCTTCTCGCT
TCCGGCGGCATCGGGATGCCCGCGTTGCAGGCCATGCTGTCCAGGCAGGTAG
ATGACGACCATCAGGGACAGCTTCAAGGATCGCTCGCGGCTCTTACCAGCCT
AACTTCGATCATTGGACCGCTGATCGTCACGGCGATTTATGCCGCCTCGGCGA
GCACATGGAACGGGTTGGCATGGATTGTAGGCGCCGCCCTATACCTTGTCTG
CCTCCCCGCGTTGCGTCGCGGTGCATGGAGCCGGGCCACCTCGACCTGAATG
GAAGCCGGCGGCACCTCGCTAACGGATTCACCACTCCAAGAATTGGAGCCAA
TCAATTCTTGCGGAGAACTGTGAATGCGCAAACCAACCCTTGGCAGAACATA
TCCATCGCGTCCGCCATCTCCAGCAGCCGCACGCGGCGCATCTCGGGCAGCG
TTGGGTCCTGGCCACGGGTGCGCATGATCGTGCTCCTGTCGTTGAGGACCCGG
CTAGGCTGGCGGGGTTGCCTTACTGGTTAGCAGAATGAATCACCGATACGCG
AGCGAACGTGAAGCGACTGCTGCTGCAAAACGTCTGCGACCTGAGCAACAAC
ATGAATGGTCTTCGGTTTCCGTGTTTCGTAAAGTCTGGAAACGCGGAAGTCAG
CGCCCTGCACCATTATGTTCCGGATCTGCATCGCAGGATGCTGCTGGCTACCC
TGTGGAACACCTACATCTGTATTAACGAAGCGCTGGCATTGACCCTGAGTGA
TTTTTCTCTGGTCCCGCCGCATCCATACCGCCAGTTGTTTACCCTCACAACGTT
CCAGTAACCGGGCATGTTCATCATCAGTAACCCGTATCGTGAGCATCCTCTCT
CGTTTCATCGGTATCATTACCCCCATGAACAGAAATCCCCCTTACACGGAGGC
ATCAGTGACCAAACAGGAAAAAACCGCCCTTAACATGGCCCGCTTTATCAGA
AGCCAGACATTAACGCTTCTGGAGAAACTCAACGAGCTGGACGCGGATGAAC
AGGCAGACATCTGTGAATCGCTTCACGACCACGCTGATGAGCTTTACCGCAG
CTGCCTCGCGCGTTTCGGTGATGACGGTGAAAACCTCTGACACATGCAGCTC
CCGGAGACGGTCACAGCTTGTCTGTAAGCGGATGCCGGGAGCAGACAAGCCC
GTCAGGGCGCGTCAGCGGGTGTTGGCGGGTGTCGGGGCGCAGCCATGACCCA
GTCACGTAGCGATAGCGGAGTGTATACTGGCTTAACTATGCGGCATCAGAGC
AGATTGTACTGAGAGTGCACCATATGCGGTGTGAAATACCGCACAGATGCGT
AAGGAGAAAATACCGCATCAGGCGCTCTTCCGCTTCCTCGCTCACTGACTCG
CTGCGCTCGGTCGTTCGGCTGCGGCGAGCGGTATCAGCTCACTCAAAGGCGG
TAATACGGTTATCCACAGAATCAGGGGATAACGCAGGAAAGAACATGTGAG
CAAAAGGCCAGCAAAAGGCCAGGAACCGTAAAAAGGCCGCGTTGCTGGCGT
TTTTCCATAGGCTCCGCCCCCCTGACGAGCATCACAAAAATCGACGCTCAAG
TCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCT
GTCCGCCTTTCTCCCTTCGGGAAGCGTGGCGCTTTCTCATAGCTCACGCTGTA
GGTATCTCAGTTCGGTGTAGGTCGTTCGCTCCAAGCTGGGCTGTGTGCACGAA
CCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTGGC
CTAACTACGGCTACACTAGAAGGACAGTATTTGGTATCTGCGCTCTGCTGAA
GCCAGTTACCTTCGGAAAAAGAGTTGGTAGCTCTTGATCCGGCAAACAAACC
ACCGCTGGTAGCGGTGGTTTTTTTGTTTGCAAGCAGCAGATTACGCGCAGAA
AAAAAGGATCTCAAGAAGATCCTTTGATCTTTTCTACGGGGTCTGACGCTCAG
TGGAACGAAAACTCACGTTAAGGGATTTTGGTCATGAGATTATCAAAAAGGA
TCTTCACCTAGATCCTTTTAAATTAAAAATGAAGTTTTAAATCAATCTAAAGT
ATATATGAGTAAACTTGGTCTGACAGTTACCAATGCTTAATCAGTGAGGCAC
CTATCTCAGCGATCTGTCTATTTCGTTCATCCATAGTTGCCTGACTCCCCGTCG
TGTAGATAACTACGATACGGGAGGGCTTACCATCTGGCCCCAGTGCTGCAAT
GATACCGCGAGACCCACGCTCACCGGCTCCAGATTTATCAGCAATAAACCAG
CCAGCCGGAAGGGCCGAGCGCAGAAGTGGTCCTGCAACTTTATCCGCCTCCA
TCCAGTCTATTAATTGTTGCCGGGAAGCTAGAGTAAGTAGTTCGCCAGTTAAT
AGTTTGCGCAACGTTGTTGCCATTGCTGCAGGCATCGTGGTGTCACGCTCGTC
GTTTGGTATGGCTTCATTCAGCTCCGGTTCCCAACGATCAAGGCGAGTTACAT
GATCCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACTGC
ATAATTCTCTTACTGTCATGCCATCCGTAAGATGCTTTTCTGTGACTGGTGAG
TACTCAACCAAGTCATTCTGAGAATAGTGTATGCGGCGACCGAGTTGCTCTTG
CCCGGCGTCAACACGGGATAATACCGCGCCACATAGCAGAACTTTAAAAGTG
CTCATCATTGGAAAACGTTCTTCGGGGCGAAAACTCTCAAGGATCTTACCGCT
GTTGAGATCCAGTTCGATGTAACCCACTCGTGCACCCAACTGATCTTCAGCAT
CTTTTACTTTCACCAGCGTTTCTGGGTGAGCAAAAACAGGAAGGCAAAATGC
CGCAAAAAAGGGAATAAGGGCGACACGGAAATGTTGAATACTCATACTCTTC
CTTTTTCAATATTATTGAAGCATTTATCAGGGTTATTGTCTCATGAGCGGATA
CATATTTGAATGTATTTAGAAAAATAAACAAATAGGGGTTCCGCGCACATTT
CCCCGAAAAGTGCCACCTGACGTCTAAGAAACCATTATTATCATGACATTAA
CCTATAAAAATAGGCGTATCACGAGGCCCTTTCGTCTTCAAGAA
OmpA Suberites domuncula Silicatein in pRHA113
Promoter-RBS-OmpATsilSilicatein-DoubleTerminator
Nucleotide
coding sequence for OmpA is bold, coding sequence for TsilSilicatein is underlined
(SEQ ID NO: 16)
tttacactttatgcttccggctcgtataatgtgtggaattgtgagcggataacaatttcagAATTCtactag
agaaagaggagaaatactagatgaaggctacaaaattggtgttgggagcggtaatcctgggttcgactctgttggcaggct
gttcttccaatgcgaagatcgaccagttatccagtgatgtggggatcaacccatacgtcggtttcgagatgggttatgact
ggctgggccgcatgccatataagggatccgtggaaaacggcgcatataaggcacaaggagtccaattgaccgcaaagt
tgggttaccccattactgatgatttggacatctacacgcgtctgggtgggatggtgtggcgcgcagacacaaaaagtaat
gtttatggtaagaatcacgatacaggagtttctcccgtttttgctggaggagtggagtatgcgatcacgccggaaatcgcg
acgcgcttggagtaccaatggactaacaacattggcgacgcgcacacgattggcactcgtcctgacaacggcatcccag
gtatgtatctcggcacgttggttgttttgtgtgttttgggggctgctattggagagccaatgcctcagtatgagttcaaggaggaatg
gcagctgtggaagaaacaacatgacaagtcttacagcaccaacttggaggaactggagaaacatcttgtctggctctccaacaa
gaagtacattgaactgcacaatgccaatgcagacacctttggattcactctagctatgaaccatctaggagatatgactgaccatg
aatacaaggagagatacctcacatacactaacagcaaatctggtaactacaccaaggtgttcaaacgtgagccatggatggcct
acccggagactgtagattggagaacaaagggcgctgtgactggtatcaagagccagggagattgtggtgccagctatgcattc
agtgccatgggtgcacttgaaggaatcaatgcacttgctactggaaagctgacctatctcagtgaacagaacatcattgattgctct
gtaccttatggtaaccatggttgcaagggtggaaacatgtatgtggctttcctctatgttgttgctaacgaaggagttgatgatgggg
gttcctatccatttagaggaaagcaatccagttgtacgtatcaagagcagtaccgtggtgcaagtatgtctggctcagttcaaatca
acagtggtagtgaatctgatctggaagcagctgtagccaatgttggtccagttgcagtagctattgatggagagtcaaatgctttca
gattctattacagtggagtgtacgactcctccagatgttctagtagcagtctcaaccacgccatggtgatcactggctatggaatttc
aaataaccaggaatactggcttgcaaagaacagctggggtgagaactggggagaactgggctatgtgaagatggccaggaac
aagtacaatcaatgtgggattgctagtgatgcctcctaccccactctctagCtcgagtaaggatctccaggcatcaaataaaacg
aaaggctcagtcgaaagactgggcctttcgttttatctgttgtttgtcggtgaacgctctctactagagtcacactggctcaccttcg
ggtgggcctttctgcg
Amino Acid
OmpA sequence is bold, TsilSilicatein sequence is underlined
(SEQ ID NO: 17)
MKATKLVLGAVILGSTLLAGCSSNAKIDQLSSDVGINPYVGFE
MGYDWLGRMPYKGSVENGAYKAQGVOLTAKLGYPITDDLDIYTRLGGMV
WRADTKSNVYGKNHDTGVSPVFAGGVEYAITPEIATRLEYQWTNNIGDAHT
IGTRPDNGIPGMYLGTLVVLCVLGAAIGEPMPQYEFKEEWQLWKKQHDKSYST
NLEELEKHLVWLSNKKYIELHNANADTFGFTLAMNHLGDMTDHEYKERYLTYT
NSKSGNYTKVFKREPWMAYPETVDWRTKGAVTGIKSQGDCGASYAFSAMGAL
EGINALATGKLTYLSEQNIIDCSVPYGNHGCKGGNMYVAFLYVVANEGVDDGG
SYPFRGKQSSCTYQEQYRGASMSGSVQINSGSESDLEAAVANVGPVAVAIDGES
NAFRFYYSGVYDSSRCSSSSLNHAMVITGYGISNNQEYWLAKNSWGENWGELG
YVKMARNKYNQCGIASDASYPTL
Full Vector Map (pBbS5a vector backbone containing-lacI, lac UV5
promoter, RBS, OmpATsilSilicatein, double terminator, pSC101 origin, AmpR, AmpR
promoter)
coding sequence for OmpA is bold, coding sequence for
TsilSilicatein is underlined
(SEQ ID NO: 18)
gacgtcggtgcctaatgagtgagctaacttacattaattgcgttgcgctcactgcccgctttccagtcggga
aacctgtcgtgccagctgcattaatgaatcggccaacgcgcggggagaggcggtttgcgtattgggcgccagggtggtttttctt
ttcaccagtgagacgggcaacagctgattgcccttcaccgcctggccctgagagagttgcagcaagcggtccacgctggtttgc
cccagcaggcgaaaatcctgtttgatggtggttaacggcgggatataacatgagctgtcttcggtatcgtcgtatcccactaccga
gatgtccgcaccaacgcgcagcccggactcggtaatggcgcgcattgcgcccagcgccatctgatcgttggcaaccagcatc
gcagtgggaacgatgccctcattcagcatttgcatggtttgttgaaaaccggacatggcactccagtcgccttcccgttccgctatc
ggctgaatttgattgcgagtgagatatttatgccagccagccagacgcagacgcgccgagacagaacttaatgggcccgctaac
agcgcgatttgctggtgacccaatgcgaccagatgctccacgcccagtcgcgtaccgtcttcatgggagaaaataatactgttga
tgggtgtctggtcagagacatcaagaaataacgccggaacattagtgcaggcagcttccacagcaatggcatcctggtcatcca
gcggatagttaatgatcagcccactgacgcgttgcgcgagaagattgtgcaccgccgctttacaggcttcgacgccgcttcgttc
taccatcgacaccaccacgctggcacccagttgatcggcgcgagatttaatcgccgcgacaatttgcgacggcgcgtgcaggg
ccagactggaggtggcaacgccaatcagcaacgactgtttgcccgccagttgttgtgccacgcggttgggaatgtaattcagctc
cgccatcgccgcttccactttttcccgcgttttcgcagaaacgtggctggcctggttcaccacgcgggaaacggtctgataagag
acaccggcatactctgcgacatcgtataacgttactggtttcacattcaccaccctgaattgactctcttccgggcgctatcatgcca
taccgcgaaaggttttgcgccattcgatggtgtccgggatctcgacgctctcccttatgcgactcctgcattaggaagcagcccag
tagtaggttgaggccgttgagcaccgccgccgcaaggaatggtgcatgcaaggagatggcgcccaacagtcccccggccac
ggggcctgccaccatacccacgccgaaacaagcgctcatgagcccgaagtggcgagcccgatcttccccatcggtgatgtcg
gcgatataggcgccagcaaccgcacctgtggcgccggtgatgccggccacgatgcgtccggcgtagaggatcgagatcgttt
aggcaccccaggctttacactttatgcttccggctcgtataatgtgtggaattgtgagcggataacaatttcagAATTCtactag
agaaagaggagaaatactagatgaaggctacaaaattggtgttgggagcggtaatcctgggttcgactctgttggcaggct
gttcttccaatgcgaagatcgaccagttatccagtgatgtggggatcaacccatacgtcggtttcgagatgggttatgact
ggctgggccgcatgccatataagggatccgtggaaaacggcgcatataaggcacaaggagtccaattgaccgcaaagt
tgggttaccccattactgatgatttggacatctacacgcgtctgggtgggatggtgtggcgcgcagacacaaaaagtaat
gtttatggtaagaatcacgatacaggagtttctcccgtttttgctggaggagtggagtatgcgatcacgccggaaatcgcg
acgcgcttggagtaccaatggactaacaacattggcgacgcgcacacgattggcactcgtcctgacaacggcatcccag
gtatgtatctcggcacgttggttgttttgtgtgttttgggggctgctattggagagccaatgcctcagtatgagttcaaggaggaatg
gcagctgtggaagaaacaacatgacaagtcttacagcaccaacttggaggaactggagaaacatcttgtctggctctccaacaa
gaagtacattgaactgcacaatgccaatgcagacacctttggattcactctagctatgaaccatctaggagatatgactgaccatg
aatacaaggagagatacctcacatacactaacagcaaatctggtaactacaccaaggtgttcaaacgtgagccatggatggcct
acccggagactgtagattggagaacaaagggcgctgtgactggtatcaagagccagggagattgtggtgccagctatgcattc
agtgccatgggtgcacttgaaggaatcaatgcacttgctactggaaagctgacctatctcagtgaacagaacatcattgattgctct
gtaccttatggtaaccatggttgcaagggtggaaacatgtatgtggctttcctctatgttgttgctaacgaaggagttgatgatgggg
gttcctatccatttagaggaaagcaatccagttgtacgtatcaagagcagtaccgtggtgcaagtatgtctggctcagttcaaatca
acagtggtagtgaatctgatctggaagcagctgtagccaatgttggtccagttgcagtagctattgatggagagtcaaatgctttca
gattctattacagtggagtgtacgactcctccagatgttctagtagcagtctcaaccacgccatggtgatcactggctatggaatttc
aaataaccaggaatactggcttgcaaagaacagctggggtgagaactggggagaactgggctatgtgaagatggccaggaac
aagtacaatcaatgtgggattgctagtgatgcctcctaccccactctctagCtcgagtaaggatctccaggcatcaaataaaacg
aaaggctcagtcgaaagactgggcctttcgttttatctgttgtttgtcggtgaacgctctctactagagtcacactggctcaccttcg
ggtgggcctttctgcgtttatacctagggtacgggttttgctgcccgcaaacgggctgttctggtgttgctagtttgttatcagaatcg
cagatccggcttcagccggtttgccggctgaaagcgctatttcttccagaattgccatgattttttccccacgggaggcgtcactgg
ctcccgtgttgtcggcagctttgattcgataagcagcatcgcctgtttcaggctgtctatgtgtgactgttgagctgtaacaagttgtc
tcaggtgttcaatttcatgttctagttgctttgttttactggtttcacctgttctattaggtgttacatgctgttcatctgttacattgtcgatct
gttcatggtgaacagctttgaatgcaccaaaaactcgtaaaagctctgatgtatctatcttttttacaccgttttcatctgtgcatatgga
cagttttccctttgatatgtaacggtgaacagttgttctacttttgtttgttagtcttgatgcttcactgatagatacaagagccataaga
acctcagatccttccgtatttagccagtatgttctctagtgtggttcgttgtttttgcgtgagccatgagaacgaaccattgagatcata
cttactttgcatgtcactcaaaaattttgcctcaaaactggtgagctgaatttttgcagttaaagcatcgtgtagtgtttttcttagtccgt
tatgtaggtaggaatctgatgtaatggttgttggtattttgtcaccattcatttttatctggttgttctcaagttcggttacgagatccattt
gtctatctagttcaacttggaaaatcaacgtatcagtcgggcggcctcgcttatcaaccaccaatttcatattgctgtaagtgtttaaa
tctttacttattggtttcaaaacccattggttaagccttttaaactcatggtagttattttcaagcattaacatgaacttaaattcatcaagg
ctaatctctatatttgccttgtgagttttcttttgtgttagttcttttaataaccactcataaatcctcatagagtatttgttttcaaaagactta
acatgttccagattatattttatgaatttttttaactggaaaagataaggcaatatctcttcactaaaaactaattctaatttttcgcttgag
aacttggcatagtttgtccactggaaaatctcaaagcctttaaccaaaggattcctgatttccacagttctcgtcatcagctctctggtt
gctttagctaatacaccataagcattttccctactgatgttcatcatctgagcgtattggttataagtgaacgataccgtccgttctttcc
ttgtagggttttcaatcgtggggttgagtagtgccacacagcataaaattagcttggtttcatgctccgttaagtcatagcgactaatc
gctagttcatttgctttgaaaacaactaattcagacatacatctcaattggtctaggtgattttaatcactataccaattgagatgggct
agtcaatgataattactagtccttttcccgggtgatctgggtatctgtaaattctgctagacctttgctggaaaacttgtaaattctgcta
gaccctctgtaaattccgctagacctttgtgtgttttttttgtttatattcaagtggttataatttatagaataaagaaagaataaaaaaag
ataaaaagaatagatcccagccctgtgtataactcactactttagtcagttccgcagtattacaaaaggatgtcgcaaacgctgtttg
ctcctctacaaaacagaccttaaaaccctaaaggcttaagtagcaccctcgcaagctcgggcaaatcgctgaatattccttttgtct
ccgaccatcaggcacctgagtcgctgtctttttcgtgacattcagttcgctgcgctcacggctctggcagtgaatgggggtaaatg
gcactacaggcgccttttatggattcatgcaaggaaactacccataatacaagaaaagcccgtcacgggcttctcagggcgtttta
tggcgggtctgctatgtggtgctatctgactttttgctgttcagcagttcctgccctctgattttccagtctgaccacttcggattatccc
gtgacaggtcattcagactggctaatgcacccagtaaggcagcggtatcatcaacaggcttacccgtcttactgtccctagtgctt
ggattctcaccaataaaaaacgcccggcggcaaccgagcgttctgaacaaatccagatggagttctgaggtcattactggatcta
tcaacaggagtccaagcgagctcgtaaacttggtctgacagttaccaatgcttaatcagtgaggcacctatctcagcgatctgtct
atttcgttcatccatagttgcctgactccccgtcgtgtagataactacgatacgggagggcttaccatctggccccagtgctgcaat
gataccgcgagacccacgctcaccggctccagatttatcagcaataaaccagccagccggaagggccgagcgcagaagtgg
tcctgcaactttatccgcctccatccagtctattaattgttgccgggaagctagagtaagtagttcgccagttaatagtttgcgcaac
gttgttgccattgctacaggcatcgtggtgtcacgctcgtcgtttggtatggcttcattcagctccggttcccaacgatcaaggcga
gttacatgatcccccatgttgtgcaaaaaagcggttagctccttcggtcctccgatcgttgtcagaagtaagttggccgcagtgttat
cactcatggttatggcagcactgcataattctcttactgtcatgccatccgtaagatgcttttctgtgactggtgagtactcaaccaag
tcattctgagaatagtgtatgcggcgaccgagttgctcttgcccggcgtcaatacgggataataccgcgccacatagcagaacttt
aaaagtgctcatcattggaaaacgttcttcggggcgaaaactctcaaggatcttaccgctgttgagatccagttcgatgtaaccca
ctcgtgcacccaactgatcttcagcatcttttactttcaccagcgtttctgggtgagcaaaaacaggaaggcaaaatgccgcaaaa
aagggaataagggcgacacggaaatgttgaatactcatactcttcctttttcaatattattgaagcatttatcagggttattgtctcatg
agcggatacatatttgaatgtatttagaaaaataaacaaataggggttccgcgcacatttccccgaaaagtgccacct
INP Suberites domuncula Silicatein in pBbS5a
Promoter-RBS-INP-Sdom Silicatein
coding sequence for INP is bold, coding sequence for SdomSilicatein is bold italics,
linker sequence is underlined
(SEQ ID NO: 19)
tttacactttatgcttccggctcgtataatgtgtggaattgtgagcggataacaatttcagAATTCtactag
agaaagaggagaaatactagatgaccttagataaagccttggtattacgcacgtgtgctaacaatatggcagatcactgcg
ggttaatttggccggccagcggtactgtggagagccgctattggcagagtacccgccgccacgaaaacgggcttgttgg
ccttctgtggggagccggtacgtctgcctttctgtccgtccatgccgatgcgcgttggatcgtctgcgaagtcgccgtagcg
gacatcatttcgttggaggaaccgggcatggtcaaattcccccgcgctgaagttgttcacgtgggtgatcgcatttcagct
tcacacttcatctcggcacgccaagccgatcccgcaagcacgagcaccagcacttcaacttccaccttaacgcctatgcc
gaccgcgattccgacaccgatgccggccgttgcatcggttactctgccggtcgccgagcaggcccgccatgaagttttcg
acgtagcgagtgtttcggccgccgctgcccccgtgaatactctgcctgtcactacgcctcagaaccttcagactgcgactt
acgggagcacattatctggggataaccactctcgcttaatcttccgtttgtgggacggtaaacgttatcgccaacttgtgg
ctcgtaccggcgagaacggggttgaggctgacatcccgtactatgtgaacgaggacgacgatattgttgataagccgga
cgaggacgacgactggatcgaggtgaagggaggaggcagtggcggtggaagtatggattaccccgaggcagtggattg
gcgcactaagggggcagttactgcggtgaaggatcagggagactgtggtgcatcatacgcatttagcgccatgggtgcctt
ggaaggcgcgaacgccttagcgaaaggcaacgccgtatccctgtcggagcaaaacatcattgattgttcaattccttatggt
aaccatggttgtcatggtggaaacatgtatgatgctttcttatacgtcatcgcgaatgagggagtggaccaagattcagcgtat
cctttcgtcgggaaacagtcctcttgtaactacaactcgaagtataagggcacgtcgatgagtggtatggtgtctattaagtcg
ggatcagagtccgatttgcaagcagccgtctctaatgtcggcccagtttcggtggcaattgacggcgcaaactcggccttccg
cttttattattctggcgtctatgattccagccgttgttctagttcgtctttgaatcatgcaatggtggtgacggggtacggcagttac
aatggaaaaaaatactggcttgccaagaactcctggggcactaactgggggaactcagggtacgttatgatggctcgtaat
aaatacaatcagtgcggtattgcgacggacgcatcctatcctacgttgtaactcgagtaaggatctccaggcatcaaataaaac
gaaaggctcagtcgaaagactgggcctttcgttttatctgttgtttgtcggtgaacgctctctactagagtcacactggctcaccttc
gggtgggcctttctgcg
INP-Sdom Silicatein INP
sequence is bold, SdomSilicatein sequence is bold italics,
linker sequence is underlined
(SEQ ID NO: 20)
MTLDKALVLRTCANNMADHCGLIWPASGTVESRYWQSTRRH
ENGLVGLLWGAGTSAFLSVHADARWIVCEVAVADIISLEEPGMVKFPRAEV
VHVGDRISASHFISARQADPASTSTSTSTSTLTPMPTAIPTPMPAVASVTLPVA
EQARHEVFDVASVSAAAAPVNTLPVTTPQNLQTATYGSTLSGDNHSRLIFRL
WDGKRYRQLVARTGENGVEADIPYYVNEDDDIVDKPDEDDDWIEVKGGGS
GGGSMDYPEAVDWRTKGAVTAVKDQGDCGASYAFSAMGALEGANALAKGNAV
SLSEQNIIDCSIPYGNHGCHGGNMYDAFLYVIANEGVDQDSAYPFVGKQSSCNYN
SKYKGTSMSGMVSIKSGSESDLQAAVSNVGPVSVAIDGANSAFRFYYSGVYDSSR
CSSSSLNHAMVVTGYGSYNGKKYWLAKNSWGTNWGNSGYVMMARNKYNQCGI
ATDASYPTL
Full Vector Map (pBbS5a vector backbone containing-lacI, lac UV5 promoter, RBS,
INPSdomSilicatein, double terminator, pSC101 origin, AmpR, AmpR promoter)
(SEQ ID NO: 21)
gacgtcggtgcctaatgagtgagctaacttacattaattgcgttgcgctcactgcccgctttccagtcggga
aacctgtcgtgccagctgcattaatgaatcggccaacgcgcggggagaggcggtttgcgtattgggcgccagggtggtttttctt
ttcaccagtgagacgggcaacagctgattgcccttcaccgcctggccctgagagagttgcagcaagcggtccacgctggtttgc
cccagcaggcgaaaatcctgtttgatggtggttaacggcgggatataacatgagctgtcttcggtatcgtcgtatcccactaccga
gatgtccgcaccaacgcgcagcccggactcggtaatggcgcgcattgcgcccagcgccatctgatcgttggcaaccagcatc
gcagtgggaacgatgccctcattcagcatttgcatggtttgttgaaaaccggacatggcactccagtcgccttcccgttccgctatc
ggctgaatttgattgcgagtgagatatttatgccagccagccagacgcagacgcgccgagacagaacttaatgggcccgctaac
agcgcgatttgctggtgacccaatgcgaccagatgctccacgcccagtcgcgtaccgtcttcatgggagaaaataatactgttga
tgggtgtctggtcagagacatcaagaaataacgccggaacattagtgcaggcagcttccacagcaatggcatcctggtcatcca
gcggatagttaatgatcagcccactgacgcgttgcgcgagaagattgtgcaccgccgctttacaggcttcgacgccgcttcgttc
taccatcgacaccaccacgctggcacccagttgatcggcgcgagatttaatcgccgcgacaatttgcgacggcgcgtgcaggg
ccagactggaggtggcaacgccaatcagcaacgactgtttgcccgccagttgttgtgccacgcggttgggaatgtaattcagctc
cgccatcgccgcttccactttttcccgcgttttcgcagaaacgtggctggcctggttcaccacgcgggaaacggtctgataagag
acaccggcatactctgcgacatcgtataacgttactggtttcacattcaccaccctgaattgactctcttccgggcgctatcatgcca
taccgcgaaaggttttgcgccattcgatggtgtccgggatctcgacgctctcccttatgcgactcctgcattaggaagcagcccag
tagtaggttgaggccgttgagcaccgccgccgcaaggaatggtgcatgcaaggagatggcgcccaacagtcccccggccac
ggggcctgccaccatacccacgccgaaacaagcgctcatgagcccgaagtggcgagcccgatcttccccatcggtgatgtcg
gcgatataggcgccagcaaccgcacctgtggcgccggtgatgccggccacgatgcgtccggcgtagaggatcgagatcgttt
aggcaccccaggctttacactttatgcttccggctcgtataatgtgtggaattgtgagcggataacaatttcagAATTCtactag
agaaagaggagaaatactagatgaccttagataaagccttggtattacgcacgtgtgctaacaatatggcagatcactgcg
ggttaatttggccggccagcggtactgtggagagccgctattggcagagtacccgccgccacgaaaacgggcttgttgg
ccttctgtggggagccggtacgtctgcctttctgtccgtccatgccgatgcgcgttggatcgtctgcgaagtcgccgtagcg
gacatcatttcgttggaggaaccgggcatggtcaaattcccccgcgctgaagttgttcacgtgggtgatcgcatttcagct
tcacacttcatctcggcacgccaagccgatcccgcaagcacgagcaccagcacttcaacttccaccttaacgcctatgcc
gaccgcgattccgacaccgatgccggccgttgcatcggttactctgccggtcgccgagcaggcccgccatgaagttttcg
acgtagcgagtgtttcggccgccgctgcccccgtgaatactctgcctgtcactacgcctcagaaccttcagactgcgactt
acgggagcacattatctggggataaccactctcgcttaatcttccgtttgtgggacggtaaacgttatcgccaacttgtgg
ctcgtaccggcgagaacggggttgaggctgacatcccgtactatgtgaacgaggacgacgatattgttgataagccgga
cgaggacgacgactggatcgaggtgaagggaggaggcagtggcggtggaagtatggattaccccgaggcagtggattg
gcgcactaagggggcagttactgcggtgaaggatcagggagactgtggtgcatcatacgcatttagcgccatgggtgcctt
ggaaggcgcgaacgccttagcgaaaggcaacgccgtatccctgtcggagcaaaacatcattgattgttcaattccttatggt
aaccatggttgtcatggtggaaacatgtatgatgctttcttatacgtcatcgcgaatgagggagtggaccaagattcagcgtat
cctttcgtcgggaaacagtcctcttgtaactacaactcgaagtataagggcacgtcgatgagtggtatggtgtctattaagtcg
ggatcagagtccgatttgcaagcagccgtctctaatgtcggcccagtttcggtggcaattgacggcgcaaactcggccttccg
cttttattattctggcgtctatgattccagccgttgttctagttcgtctttgaatcatgcaatggtggtgacggggtacggcagttac
aatggaaaaaaatactggcttgccaagaactcctggggcactaactgggggaactcagggtacgttatgatggctcgtaat
aaatacaatcagtgcggtattgcgacggacgcatcctatcctacgttgtaaCtcgagtaaggatctccaggcatcaaataaaa
cgaaaggctcagtcgaaagactgggcctttcgttttatctgttgtttgtcggtgaacgctctctactagagtcacactggctcacctt
cgggtgggcctttctgcgtttatacctagggtacgggttttgctgcccgcaaacgggctgttctggtgttgctagtttgttatcagaat
cgcagatccggcttcagccggtttgccggctgaaagcgctatttcttccagaattgccatgattttttccccacgggaggcgtcact
ggctcccgtgttgtcggcagctttgattcgataagcagcatcgcctgtttcaggctgtctatgtgtgactgttgagctgtaacaagtt
gtctcaggtgttcaatttcatgttctagttgctttgttttactggtttcacctgttctattaggtgttacatgctgttcatctgttacattgtcg
atctgttcatggtgaacagctttgaatgcaccaaaaactcgtaaaagctctgatgtatctatcttttttacaccgttttcatctgtgcatat
ggacagttttccctttgatatgtaacggtgaacagttgttctacttttgtttgttagtcttgatgcttcactgatagatacaagagccata
agaacctcagatccttccgtatttagccagtatgttctctagtgtggttcgttgtttttgcgtgagccatgagaacgaaccattgagat
catacttactttgcatgtcactcaaaaattttgcctcaaaactggtgagctgaatttttgcagttaaagcatcgtgtagtgtttttcttagt
ccgttatgtaggtaggaatctgatgtaatggttgttggtattttgtcaccattcatttttatctggttgttctcaagttcggttacgagatc
catttgtctatctagttcaacttggaaaatcaacgtatcagtcgggcggcctcgcttatcaaccaccaatttcatattgctgtaagtgtt
taaatctttacttattggtttcaaaacccattggttaagccttttaaactcatggtagttattttcaagcattaacatgaacttaaattcatc
aaggctaatctctatatttgccttgtgagttttcttttgtgttagttcttttaataaccactcataaatcctcatagagtatttgttttcaaaag
acttaacatgttccagattatattttatgaatttttttaactggaaaagataaggcaatatctcttcactaaaaactaattctaatttttcgct
tgagaacttggcatagtttgtccactggaaaatctcaaagcctttaaccaaaggattcctgatttccacagttctcgtcatcagctctc
tggttgctttagctaatacaccataagcattttccctactgatgttcatcatctgagcgtattggttataagtgaacgataccgtccgtt
ctttccttgtagggttttcaatcgtggggttgagtagtgccacacagcataaaattagcttggtttcatgctccgttaagtcatagcga
ctaatcgctagttcatttgctttgaaaacaactaattcagacatacatctcaattggtctaggtgattttaatcactataccaattgagat
gggctagtcaatgataattactagtccttttcccgggtgatctgggtatctgtaaattctgctagacctttgctggaaaacttgtaaatt
ctgctagaccctctgtaaattccgctagacctttgtgtgttttttttgtttatattcaagtggttataatttatagaataaagaaagaataaa
aaaagataaaaagaatagatcccagccctgtgtataactcactactttagtcagttccgcagtattacaaaaggatgtcgcaaacg
ctgtttgctcctctacaaaacagaccttaaaaccctaaaggcttaagtagcaccctcgcaagctcgggcaaatcgctgaatattcct
tttgtctccgaccatcaggcacctgagtcgctgtctttttcgtgacattcagttcgctgcgctcacggctctggcagtgaatgggggt
aaatggcactacaggcgccttttatggattcatgcaaggaaactacccataatacaagaaaagcccgtcacgggcttctcagggc
gttttatggcgggtctgctatgtggtgctatctgactttttgctgttcagcagttcctgccctctgattttccagtctgaccacttcggatt
atcccgtgacaggtcattcagactggctaatgcacccagtaaggcagcggtatcatcaacaggcttacccgtcttactgtccctag
tgcttggattctcaccaataaaaaacgcccggcggcaaccgagcgttctgaacaaatccagatggagttctgaggtcattactgg
atctatcaacaggagtccaagcgagctcgtaaacttggtctgacagttaccaatgcttaatcagtgaggcacctatctcagcgatct
gtctatttcgttcatccatagttgcctgactccccgtcgtgtagataactacgatacgggagggcttaccatctggccccagtgctg
caatgataccgcgagacccacgctcaccggctccagatttatcagcaataaaccagccagccggaagggccgagcgcagaa
gtggtcctgcaactttatccgcctccatccagtctattaattgttgccgggaagctagagtaagtagttcgccagttaatagtttgcg
caacgttgttgccattgctacaggcatcgtggtgtcacgctcgtcgtttggtatggcttcattcagctccggttcccaacgatcaag
gcgagttacatgatcccccatgttgtgcaaaaaagcggttagctccttcggtcctccgatcgttgtcagaagtaagttggccgcag
tgttatcactcatggttatggcagcactgcataattctcttactgtcatgccatccgtaagatgcttttctgtgactggtgagtactcaa
ccaagtcattctgagaatagtgtatgcggcgaccgagttgctcttgcccggcgtcaatacgggataataccgcgccacatagca
gaactttaaaagtgctcatcattggaaaacgttcttcggggcgaaaactctcaaggatcttaccgctgttgagatccagttcgatgt
aacccactcgtgcacccaactgatcttcagcatcttttactttcaccagcgtttctgggtgagcaaaaacaggaaggcaaaatgcc
gcaaaaaagggaataagggcgacacggaaatgttgaatactcatactcttcctttttcaatattattgaagcatttatcagggttattg
tctcatgagcggatacatatttgaatgtatttagaaaaataaacaaataggggttccgcgcacatttccccgaaaagtgccacct

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A genetically modified host cell comprising a nucleic acid encoding a silicatein fusion peptide.

2. The cell of claim 1, wherein the silicatein fusion peptide comprises one or more of the group selected from:
a) a fusion of silicatein and at least one selected from the group consisting of a membrane protein and a transmembrane protein domain;
b) a sequence selected from the group consisting of SEQ ID NO:2 and SED ID NO: 4, or a fragment or variant thereof; and
c) a sequence selected from the group consisting of: SEQ ID NO: 14, SEQ ID NO:17 and SEQ ID NO:20.

3. The cell of claim 1, wherein the cell is selected from the group of: a bacterial cell and an E. coli cell.

4-5. (canceled)

6. The cell of claim 1, wherein the nucleic acid molecule encoding the silicatein fusion peptide comprises a nucleotide sequence selected from the group consisting of SEQ ID NO: 1, SEQ ID NO:3, SEQ ID NO:13, SEQ ID NO: 16 and SEQ ID NO: 19.

7. The cell of claim 2, wherein the membrane protein comprises one or more is selected from the group consisting of:
a) OmpA, OmpC, OmpF, TolC, YaeT and RsaA, or a homolog or ortholog thereof; and
b) a sequence as set forth in SEQ ID NO:6, or a fragment or variant thereof.

8. (canceled)

9. The cell of claim 7, wherein the nucleic acid molecule encoding the membrane protein comprises a sequence as set forth in SEQ ID NO:5.

10. The cell of claim 2, wherein the transmembrane protein domain is (a) an ice nucleation protein (INP) transmembrane domain or (b) an INP transmembrane domain comprising a sequence as set forth in SEQ ID NO:8, or a fragment or variant thereof.

11. (canceled)

12. The cell of claim 10, wherein the nucleic acid molecule encoding the INP transmembrane domain comprises a sequence as set forth in SEQ ID NO:7.

13-14. (canceled)

15. The cell of claim 1, wherein the nucleic acid encoding the silicatein fusion peptide is operatively linked to at least one regulatory element selected from the group consisting of an inducible promoter or a variant thereof, a ribosome binding domain (RBD), a transcriptional termination element, and a double transcriptional termination element.

16. The cell of claim 1, wherein the nucleic acid is operatively linked to an inducible promoter, wherein (a) the inducible promoter is selected from the group consisting of a lacUV5 promoter and a rhaB promoter or (b) the presence of at least one inducer molecule specific for the induction of the inducible promoter causes the expression of the silicatein fusion peptide.

17. The cell of claim 15, wherein the nucleic acid molecule encoding the silicatein fusion peptide comprises a sequence selected from the group consisting of SEQ ID NO: 15, SEQ ID NO: 18 and SEQ ID NO:21.

18. (canceled)

19. The cell of claim 1, further comprising a nucleic acid molecule encoding at least one molecule to alter cellular morphology.

20. The cell of claim 19, wherein the molecule to alter cellular morphology is selected from the group consisting of BolA, SulA and CrvAB.

21. A system for producing a bioglass microlens, the system comprising
a) the cell of claim 1; and
b) a silicate molecule, sodium silicate solution, orthosilicate, tetraethyl orthosilicate (TEOS), tetramethyl orthosilicate, tetrabutyl orthosilicate, or salt, derivative or analog thereof.

22. (canceled)

23. The system of claim 21, wherein the system further comprises a molecule for induction of expression of the silicatein fusion peptide from an inducible promoter, the molecule being selected from one or more of the group consisting of: an inducer molecule, IPTG, and rhamnose.

24. (canceled)

25. A method of producing a bioglass microlens, the method comprising contacting the cell of claim 1 with a silicate molecule, sodium silicate solution, orthosilicate, tetraethyl orthosilicate (TEOS), tetramethyl orthosilicate, tetrabutyl orthosilicate, or salt, derivative or analog thereof.

26. (canceled)

27. The method of claim 25, wherein the method further comprises contacting the cell with a molecule for induction of expression of the silicatein fusion peptide from an inducible promoter, the molecule being selected from one or more of the group consisting of: an inducer molecule, IPTG, and rhamnose.

28. (canceled)

29. A product generated according to the method of claim 25, wherein the product is selected from one or more of the group consisting of:
a) a bioglass microlens;
b) a bioglass microlens, wherein the microlens comprises a polysilicate layer about 15 nm-25 nm thick, wherein the polysilicate is in a spherical, cylindrical or ovoid formation having a length of about 0.5 μm-3 μm and a radius of about 0.25 μm-1 μm;
c) a device comprising at least one bioglass microlens;
d) a device comprising an array of bioglass microlenses; and
e) a device comprising at least one bioglass microlens, wherein the device is selected from the group consisting of an optical device, a solar panel, a solar cell, a compact image sensor, a 3D optical display, a photovoltaic concentrator, a flexible image sensor, a flexible substrate with optical activity, a one-photon microscope, a multiphoton microscope, and a super-resolution emission depletion microscope (STED).

30-33. (canceled)

34. The method of claim 25, further comprising coating a surface or substrate with the cell.

35-37. (canceled)

38. A method of capturing and focusing light comprising the steps of:
providing the product of claim 29; and
capturing and focusing light with the product.
